(19) United States
(12) Patent Application Publication
(10) Pub. No.: US 2022/0079993 A1
(43) Pub. Date: Mar. 17, 2022

(54) NON-NATURALLY OCCURRING THERMOGENIC ADIPOCYTES, METHODS OF MAKING, AND METHODS OF USE THEREOF

(71) Applicant: University of Georgia Research Foundation, Inc., Athens, GA (US)

(72) Inventors: Stephen Dalton, Athens, GA (US); Amar Meghnauth Singh, Athens, GA (US); John Welch Avery, Snellville, GA (US); Liang Zhang, Athens, GA (US)

(21) Appl. No.: 17/420,484

(22) PCT Filed: Jan. 8, 2020
(86) PCT No.: PCT/US2020/012728

§ 371 Date: Jul. 2, 2021

Related U.S. Application Data
(60) Provisional application No. 62/789,892, filed on Jan. 8, 2019.

(51) Int. Cl. A61K 35/35; A61P 3/06; C12N 5/0775; C12N 5/00; A61P 3/10; A61P 3/04

(52) U.S. Cl. CPC A61K 35/35; A61P 3/06; C12N 5/0667; C12N 5/0031; A61P 3/10; C12N 2500/32; C12N 2500/44; C12N 2501/115; C12N 2501/105; C12N 2501/155; A61P 3/04

(57) ABSTRACT

Non-naturally occurring thermogenic adipocytes are provided. The cells have a distinctive molecular signature, and can be distinguished from predecessor cells such as adipose-derived stem cells as well as other naturally occurring and induced thermogenic cells. Differentiation media that can induce differentiation of ADSC and white adipocytes into thermogenic adipocytes is also provided. Methods of making thermogenic adipocytes, thermogenic adipocytes made according the disclosed methods as well as conditioned media made according a method of incubating the cells in a tissue culture media are also provided. Compositions including thermogenic adipocytes, conditioned media, secreted factor(s), active agents that increase the number or activity of thermogenic adipocytes, or a combination thereof are also disclosed and can be used to treat a variety of diseases and conditions, particularly obesity and metabolic disorders.

### CROSS-REFERENCE TO RELATED APPLICATIONS

This application claims the benefit of and priority to U.S. Provisional Application No. 62/789,892 filed Jan. 8, 2019, which is hereby incorporated by reference in its entirety.

### FIELD OF THE INVENTION

The field of the invention generally relates to compositions and methods for treating and preventing metabolic and weight-related conditions.

### BACKGROUND OF THE INVENTION

Thermogenic (brown, beige or brite) adipocytes have therapeutic potential for the treatment of obesity-associated diseases, such as type 2 diabetes (Harms and Seale, Nat Med 19, 1252-1263 (2013); Lidell et al., J Intern Med 276, 364-377 (2014); Singh and Dalton, Trends Endocrinol Metab 29, 349-359 (2018)). This is evident by observation that lean adults have significant brown adipose tissue (BAT) depots, whereas individuals with a high body mass index have little or no BAT (Cypess et al., N Engl J Med 360, 1509-1517 (2009); Saito et al., Diabetes 58, 1526-1531 (2009); van Marken Lichtenbelt et al., N Engl J Med 360, 1500-1508 (2009); Virtanen et al., N Engl J Med 360, 1518-1525 (2009)). In animal models, transplantation of mouse and human brown or beige adipocytes reduce obesity and hyperglycemia, and may therefore offer a therapeutic strategy to intervene in metabolic diseases in patients (Gunawardana and Piston, Diabetes 61, 674-682 (2012); Hepler et al., Elife 6, (2017); Liu et al., Cell Res 23, 851-854 (2013); Min et al., Nat Med 22, 312-318 (2016); Nishio et al., Cell Metab 16, 394-406 (2012); Stanford et al., J Clin Invest 123, 215-223 (2013)).

Perhaps the largest obstacle preventing the use of thermogenic adipocytes in a therapeutic setting is developing a source of the cells with high efficiency, purity and at sufficient scale for development as a therapeutic product (Singh and Dalton, Trends Endocrinol Metab 29, 349-359 (2018)).

Addressing this “cell source” problem was attempted using several models, but none are suitable for further clinical development because of efficiency issues and lack of in vivo functional validation (Mukherjee, et al., Curr Diabetes Rev 12, 414-428 (2016)). For example, pluripotent stem cells (PSCs) used to generate thermogenic adipocytes, through a dermomyotome progenitor or mesenchymal progenitor (Guenantin et al., Diabetes 66, 1470-1478 (2017); Hafner et al., Sci Rep 6, 32490 (2016); Nishio et al., Cell Metab 16, 394-406 (2012)), offer the advantage of potential scale-ability to generate the thermogenic adipocytes. However, the efficiency, reproducibility and time-frame of generating PSC-derived thermogenic adipocytes with existing technologies makes them unviable for therapeutic development. Other approaches used genetic manipulation to ectopically express transcription factors to drive differentiation or reprogram cells to a thermogenic identity (Ahfeldt et al., Nat Cell Biol 14, 209-219 (2012); Kishida et al., Stem Cell Reports 5, 569-581 (2015)). While these approaches may be useful for drug discovery, there are issues relating to scale and purity that make this approach unsuitable for therapeutic use. Moreover, genetically manipulated cells can pose safety issues and may have difficulty passing regulatory scrutiny.

BMP4 and BMP7 have been used to induce the white-to-brown transition of primary human adipose stem cells (Elsen, et al., Am J Physiol Cell Physiol, 306: C431-C440 (2014)), while BMP4 alone can induce adipocyte commitment while inhibiting the acquisition of a brown phenotype during terminal differentiation (Modica, et al., Cell Reports, 16, 2243-2258 (2016)). Adipose-derived Stem Cells (ADSCs) were also found to generate beige adipocytes (Bartesaghi et al., Mol Endocrinol 29, 130-139 (2015), Wang, et al., Biochem Biophys Res Commun 478, 689-695 (2016)), although efficiency and reproducibility of these finding has been controversial (Zhang, et al., Cell Physiol Biochem 51, 2900-2915 (2018)). The inefficient conversion of ADSCs to beige cells and absence of in vivo data supporting a therapeutic role, are major barriers that need to be overcome for this approach to have clinical utility. There remains a need for improved therapeutic options for obesity-related conditions.

Thus, it is an object of the invention to provide compositions and methods for treating and preventing metabolic and weight-related conditions.

### SUMMARY OF THE INVENTION

Non-naturally occurring thermogenic adipocytes are provided. The cells have a distinctive molecular signature. For example, the thermogenic adipocytes are typically characterized by increased uncoupling protein 1 (UCP1) expression, increased Type II iodothyronine deiodinase (DIO2) expression, or a combination thereof relative to adipose-derived stem cells (ADSC), white adipocytes (WAT), or a combination thereof. In some embodiments, the disclosed thermogenic adipocytes can be distinguished from naturally occurring and other induced thermogenic adipocytes including brown adipocytes (BAT) and beige cells by having, for example, a reduced Intercellular Adhesion Molecule 1 (ICAM1) expression, increased matrix metalloproteinase-3 (MMP3) expression, or a combination relative thereto. Thus, in some embodiments, the cells have increased UCP1 and DIO2 expression. In some embodiments, the cells have reduced ICAM1 and increased MMP3 expression. The increased or reduced expression can be mRNA expression, protein expression, or the combination thereof. In some embodiments, contacting the cells with forskolin can further increase UCP1 expression, DIO2 expression, or the combination thereof.

The cells can also have a functional signature characteristic of thermogenic adipocytes. For example, levels of mitochondrial respiration, glycolysis, or a combination thereof can be increased in the cells relative to WAT. The respiration can be basal respiration, maximal respiration, or a combination thereof, and can be uncoupled respiration (proton leak). In some embodiments, contacting the cells with forskolin further increases the respiration, glycolysis, or combination thereof in the cells. Forskolin may also induce or increase lipolysis and/or activate a thermogenic program. The lipolysis may be characterized by an increase in extracellular glycerol. Activation of a thermogenic program may be identified by, for example, an increase in the cells' ability to reduce signal intensity of a thermogenic dye. In some embodiments, the cells can elevate energy expenditure and/or oxygen consumption in a subject following transplantation therein, preferably without a change in the respiratory exchange ratio and/or locomotor activity in the subject. In a particular embodiment having most or all of the molecular criteria (e.g., increased UDP1, DIO2, and MMP3, and decreased ICAM1) and functional criteria discussed above, the cells can be referred to Glucocytes (GC) or beige cells (e.g., ADSC-derived beige cells, non-naturally occurring beige cells, etc).

Differentiation media that can induce differentiation of ADSC and white adipocytes into thermogenic adipocytes is also provided. The media typically includes one or more additives of Table 1 (below) in combination with one or more of the growth factors and inhibitors of Table 2 (below) in a base media composed of a balanced salt solution, amino acids, and vitamins, wherein the media can induce differentiation of ADSC, WAT, or both into thermogenic adipocytes. In some embodiments, the differentiated thermogenic adipocytes have some or all of the molecular and/or functional signature of the non-naturally occurring thermogenic adipocytes disclosed above and elsewhere herein. In some embodiments, the differentiation media includes all of the additives of Table 1, optionally at the concentrations according to Table 1. In some embodiments, the differentiation media includes all of the factors and inhibitors of Table 2, optionally in the concentrations according to Table 2. In specific embodiments, a cell culture media includes the ingredients in Table 3 (below) or Table 4 (below) optionally at the concentrations according to Table 3 or Table 4, respectively.

Methods of making thermogenic adipocytes are disclosed. The methods typically include incubating ADSC or WAT with the disclosed differentiation media until the cells differentiate into thermogenic adipocytes. In some embodiments, the cells are incubated for at least 3, 7, 10, 14, 18, or 21 days. The media can be changed, for example, every few days.

Thermogenic adipocytes made according the disclosed methods as well as conditioned media made according to a method of incubating the cells in a tissue culture media, optionally a serum-free tissue culture media, for one or more days and separating the media from the cells are also provided. One or more cell-secreted materials can be isolated from the conditioned media. In some embodiments, the cell-secreted factor(s) include or consist of one or more cytokines, cell signing proteins, extracellular vesicles, or a combination thereof. The extracellular vesicles can be, for example, exosomes. The cell-secreted factors can include or consist of adipokines.

Thermogenic adipocytes can also be used to identify active agent compounds that modulate the number and/or activity of the thermogenic adipocytes. Such compounds include, but are not limited to, Trequinisn HCL, Anagrelide, Milrinone, L-368,899, pilocarpine nitrate, LY310-762, Felodipine, Dinaciclib, AT9283, PF-04691502, PIK-75, SU 5416, and pharmaceutically acceptable salts thereof.

Compositions including thermogenic adipocytes, conditioned media, secreted factor(s), active agent modulators of thermogenic adipocytes, or a combination thereof are also provided. The compositions can include a pharmaceutically acceptable carrier. The compositions can include a delivery vehicle or depot. The delivery vehicle or depot can include a hydrogel, polymeric polymer, or collagen. The polymeric polymer can be, for example, a polyester polymer such as poly(lactic acid), poly(glycolic acid), poly(lactic-co-glycolic acid), or a blend or copolymer thereof.

Methods of treatment are also provided. The methods typically include administering an effective amount of a composition including thermogenic adipocytes, conditioned media, secreted factor(s), an active agent compounds (including, but not limited to, Trequinisn HCL, Anagrelide, Milrinone, L-368,899, pilocarpine nitrate, LY310-762, Felodipine, Dinaciclib, AT9283, PF-04691502, PIK-75, SU 5416, and pharmaceutically acceptable salts thereof), or a combination thereof, to a subject in need thereof. The composition is typically administered by transplantation, subcutaneous injection, or intravenous injection. Administration can be site specific (e.g., local). Active agent compounds can be administered by any suitable means, which in some embodiments may include oral or other routes of administration.

In some embodiments, the subject has one or more diseases, disorders, or conditions such as obesity or excessive weight gain, a metabolic disorder, vascular disease, heart disease, atherosclerosis, dyslipidemia, liver steatosis, loss of physical activity, or loss of endurance, or a symptom or comorbidity thereof associated therewith. The metabolic disorder can be or include insulin resistance, Type 1 or 2 diabetes mellitus, insulin insensitivity, impaired fasting glycaemia, impaired glucose tolerance (IGT), dysglycemia, dyslipidemia, hypertriglyceridemia, hyperglyceridemia, dyslipoproteinemia, hyperlipidemia, hypercholesterolemia, hypolipoproteinemia, or metabolic syndrome.

In some embodiments, the composition is administered to the subject in an effective amount to increase glucose uptake, increase thermogenic activity, reduce weight gain, reduce body weight, increase weight loss, increase energy expenditure, reduce hyperglycemia, increase VO₂, increase energy expenditure, increase core temperature, reduce insulin resistance, improve glucose tolerance, improve insulin sensitivity, or a combination thereof in the subject. In some embodiments, the respiration exchange ratio and/or activity levels are not significantly changed in the subject.

The methods can further include administering to the subject one or more factors to enhance angiogenic, antifibrotic, anti-apoptotic and anti-inflammatory properties. Factor(s) include, for example, VEGF, hepatic growth factor (HGF), fibroblast growth factor (FGF), transforming growth factor (TGF) β, platelet-derived growth factor (PDGF), IL-8, matrix metalloproteinase (MMP) 2, and combinations thereof.

Methods of testing the effect of compounds on the differentiation and/or activation of the disclosed non-naturally occurring thermogenic adipocytes are also provided. The methods can include, for example, adding or subtracting one or more factors from the thermogenic culture conditions or contacting the adipocytes in vitro with a test compound, and analyzing the molecular and/or functional effect of adding or subtracting the factor(s) or compound(s) on the cells. In some embodiments, the methods including determining protein expression, protein activity, or binding activity of one or more marker of adipocytes, determination of nucleic acid transcription or translation, examining cell structure and histology, respiration levels (e.g., basal respiration, maximal respiration, proton leak, etc.), or other assays discussed herein. Exemplary assays include FACS, FACE, ELISA, Northern blotting, Western blotting, qRT-PCR, RNA-Seq, immunostaining, a lipolysis assay, and/or a glycerol release assay. In some embodiments, the methods include determining the mRNA or protein levels of ICAM1, MMP3, UCP1, DIO2, or a combination thereof before and after addition or subtraction of the factor or compound. The methods can be repeated or carried out in parallel with two or more different factors or compounds. In some embodiments, the methods are a screen, for example a high through-put screen, wherein the method is carried out, for example, 5, 10, 15, 20, 25, 50, 100, or any other integer between 1 and 10,000, times.

### BRIEF DESCRIPTION OF THE DRAWINGS

FIG. 1 is a diagram illustrating exemplary applications of GlucoCytes (also referred to herein including the figures, examples and other text as beige cells (e.g., ADSC-derived beige cells, ADSC beige cells, etc.). GlucoCytes are a new cell type that can be generated from ADSCs or white adipocytes, and have broad applications for cell therapy, drug screening, and extracellular vesicles (EV)-based therapy.

FIG. 2A is a bar graph showing RNA expression levels by qRT-PCR for indicated genes (UCP1, DIO2) for adipose derived stem cells (ADSCs), GlucoCytes (GC), or GC with forskolin (FSK, 20 uM). FIG. 2B is a bar graph showing quantitation of immunostaining data (UCP1) from 6 independent fields of view. FIGS. 2C and 2D are bar graphs showing qRT-PCR of UCP1 of GC induced from an additional line purchased from ThermoFisher (2C) and an immortalized ADSC line purchased from ATCC (ASC52-telo) (2D). Transcript levels were determined by qRT-PCR. n=3 for each independent replicate. Data are mean+/−standard deviation. *P<0.0.05, **P<0.01, ***P<0.005. FIGS. 2E and 2F are bar graphs showing comparison of B8 method to methods described by Wang, et al. and Bartesaghi, et al., respectively. Differentiations were performed exactly as described according to previous publications, imaged, and samples collected for qRT-PCR at recommended days. Statistical significance is shown if B8 method is enhanced over both Wang et al23. AND Bartesaghi et al22., *P<0.05, **P<0.01, ***P<0.005. FIG. 2G-2N are bar graphs showing transcriptional analysis of adipocyte markers (UCP1 (2G), ADIPOQ (2H), PPARGC1 (2I), PPARG (2J), PLIN1 (2K), CIDEA (2L), PDK4 (2M), CEBPA (2N)) during beige adipocyte differentiation in B8 medium. Cell lysates were collected at indicated days and RNA was isolated and analyzed by qRT-PCR for marker expression. Each statistical measurement was compared to the preceding time-point (i.e. d5 was compared to ADSC, d10 was compared to d5, d20 was compared to d10). *P<0.05, **P<0.01, ***P<0.005. FIGS. 2O and 2P are bar graphs showing analysis of additional normal and type 2 diabetic (T2D) ADSC lines transcript levels of UCP1 of beige adipocytes differentiated from ADSCs (line 1, lot: 1001002) after five passages (2O) and ten passages (2P). Data are mean+/−standard deviation. *P<0.0.05, **P<0.01, ***P<0.005. FIGS. 2Q-2T are bar graphs showing differentiation of four additional lines (two male and two female) purchased from Lonza of varying age, BMI and T2D status. Each line was differentiated for 21 days to beige adipocytes using B-8 medium and transcript levels of UCP1 (2Q), PPARGC1 (2R), IRF4 (2S), and ADIPOQ (2T) were measured by Taqman qRT-PCR for indicated genes +/−FSK for 6 hours (20 μM).

FIG. 3A is a scatter plot comparing global transcriptomes of ADSC-derived beige adipocytes and human brown adipocytes (Shinoda, et al., Nat Med 21, 389-394 (2015)). Transcripts typically expressed in thermogenic adipocytes at elevated levels are indicated. Middle segment data points represent a <2-fold difference between data sets, top segment data points represent a >2-fold increase in this study and bottom segment data points represent a >2-fold increase in brown adipocytes. Forskolin, FSK. FIG. 3B is a hierarchical clustering dendrogram comparing GC to other cell types. FIG. 3C is a scatter plot of RNA-seq of GC, before and after FSK. FIG. 3D is a scatter plot of BAT (from Inagaki, et al., Nat Rev Mol Cell Biol 17, 480-495 (2016)), before and after FSK. FIG. 3E is a heat map showing RNA-seq analysis following treatment of white adipocytes to GC after 2 weeks of differentiation in B-8 media, and their subsequent induction with forskolin (FSK, 20 uM). FIG. 3F is a hierarchical clustering dendrogram comparing ADSC-derived beige adipocytes to other primary human cell types. Boxes indicate cell types with similar Euclidian distances. FIG. 3G is a linear regression analysis comparing data from this study with beige adipocytes (FSK-treated and Shinoda et al. FSK-treated) indicating a similar stimulation response. FIG. 3H is a heatmap of RNA-seq data focusing on transcripts typically enriched in thermogenic adipocytes. ADSCs, white adipocytes and ADSC-derived beige adipocytes −/+forskolin, FSK (20 μM) are shown. FIG. 3I is a plot of a ProFAT (Cheng, et al., Cell Rep 23, 3112-3125 (2018)) computational analysis from reference datasets that integrates 97 human brown and white adipocyte samples, compared to the RNA-seq from ADSC-derived beige adipocytes. The browning probability score was determined from the correlation between the reference datasets and the ADSC-derived beige adipocyte dataset. Data for beige, white (WA) and brown (BA) adipocytes were analyzed through this pipeline. FIG. 3J is a heatmap from ProFAT37 analysis comparing indicated samples by adipose transcripts. From a total of 97 human adipocyte RNA-seq and microarray datasets, transcriptional profiles were aggregated together and following the removal of batch effects using a computational algorithm, normalized gene expression values were calculated and used for BAT or WAT references. These BAT and WAT reference aggregates were compared to BA33 or ADSC-derived beige adipocytes (this study). A total of 49 genes, indicated in the heatmap, were used to interrogate RNA-seq datasets so that a browning probability score was generated (FIG. 3F). Relative gene expression changes are indicated by the Z-score. Black and gray boxes represent predicted mitochondrial or non-mitochondrial genes, respectively.

FIG. 4 is a heatmap comparing markers for GC and all other thermogenic adipocytes from in vitro or in vivo sources by RNA-sequencing.

FIG. 5A is a line graph showing the oxygen consumption rate from a Seahorse Mito Stress Test assay of GC compared to white adipocytes (WA). Cells were stimulated with forskolin (FSK, 20 uM) for 24 hours prior to treatment, and oligomycin (3 μM), FCCP (3 μM), and antimycin a/rotenone (5 μM) were added at indicated time points. n=9, where n is the number of independent replicates for each cell type/condition. Assay was normalized to total protein content. FIG. 5B is a line graph showing extracellular acidification rate from the assay in FIG. 5A. FIGS. 5C-5E are bar graphs showing basal respiration rate (5C) from the assay maximal respiration (5D) and proton leak (5E) calculated from assay performed in FIG. 5A. FIG. 5F is a bar graph showing the results of a lipolysis assay of ADSCs or GC (BA), treated with forskolin for indicated time period. FIG. 5G is a bar graph showing quantification of thermogenic dye assay of GC with or without forskolin treatment. Cells were imaged after 30 minutes of ERThermaAC dye (125 nM) treatment and then 3 hours later, with and without forskolin, to evaluate the basal and activated thermogenic states. n=24, where n is the number of independent replicates, for basal and activated (activ.) states. *P<0.05, **P<0.01.

FIGS. 6A-6I are bar graphs showing that GC transplantation increases energy expenditure, oxygen consumption and reduces body weight. FIGS. 6A and 6B are bar graphs showing energy expenditure (kcal/kg/hr) during light (6A) and dark (6B) cycles. NOD/SCID mice were transplanted with 2 million ADSCs or GC, and indirect calorimetry assays were performed. n=8, where n is the number of mice used per group in the assay.

FIGS. 6C and 6D are bar graphs showing oxygen consumptions levels (VO₂) from indirect calorimetry assay during light (6C) and dark (6D) cycles. FIGS. 6E and 6F are bar graphs showing respiratory exchange ratio from indirect calorimetry assay during light (6E) and dark (6F) cycles (no change, indicating no hyperventilation from the mice). FIGS. 6G and 6H are bar graphs showing activity levels of mice during light (6G) and dark (6H) cycles (no change, indicating that increased activity is not the cause for the increased energy expenditure or oxygen consumption). FIG. 6I is a bar graph showing core body temperature was slightly elevated from the GC transplants. FIG. 6J is a bar graph showing GC transplants led to a significant decrease in body weight (grams) over time (weeks) Mann-Whitney rank sum test; *P<0.0.5, **P<0.01, ***P<0.005.

FIG. 7A is a schematic of an experimental design for streptozotocin (STZ)-induced hyperglycemia. Times of STZ administration, cell transplantation and blood glucose sampling are indicated. FIG. 7B is a box-plot of blood glucose measurements in STZ-treated mice assayed according to FIG. 7A (n=8 per group). FIG. 7C is a bar graph showing Quantitation of PET analysis as body mass (% ID/g) of 18F-FDG treated mice at 2-weeks post transplantation of beige adipocytes compared to saline, n=4 per group. Data is graphed as the % injected dose/gram. *p<0.0.05, **p<0.01, ***p<0.005. FIG. 7D is a line graph of an intra-peritoneal glucose tolerance test (IPGTT) following transplantation (5 days) into the intrascapular, hindlimb regions, n=5 per group. FIG. 7E is a bar graph showing pre-transplant blood glucose levels for data shown in FIG. 7D, n=5 per group. FIG. 7F is a plot of thermal imaging data of mice transplanted into the intrascapular region with human beige adipocytes or human ADSCs. +/−standard deviation, n=3, per group. FIG. 7G is a line graph showing analysis of food intake following beige adipocyte transplantation. Mice (n=5 per group) were transplanted with ADSCs or beige adipocytes into the leg muscle and back and food intake was monitored daily by measuring weight. FIGS. 7H and 7I are bar graphs showing analysis of mouse adipose tissue depots following human beige tissue transplantation. Transcript levels for Ucpl, normalized to Gapdh were analyzed following transplantation of human beige tissue into the leg and back of NOD/SCID mice by Taqman qRT-PCR (n=4) per group (ADSC or Beige transplants). FIGS. 7J and 7K are graphs showing analysis of beige adipocyte transplantation under thermoneutral conditions. Beige adipocytes were transplanted into NOD-SCID mice that were previously housed under standard shelf (21° C.) or thermoneutral (30° C.) conditions for two weeks. Mice were monitored prior to transplant for non-fasting blood glucose levels (7J, bar graph)), and after 5 days, glucose measurements were taken by IPGTT (7K, line graph).

FIG. 8A is a scheme used for high-throughput screening (HTS). FIG. 8B is a scatter-plot of glycerol release for each compound assayed (n=3889) in an HTS screen. Data are shown as the percentage of glycerol released compared to untreated beige adipocytes (% of control). FIG. 8C is a computational analysis of preliminary hits identified in the HTS using BiNChE. FIG. 8D is a plot showing the results of a secondary hit validation of primary compounds performed by evaluating levels of UCP1 transcript by qRT-PCR. Unstimulated beige cells, selected adrenergic agonists, and potential new non-adrenergic beige fat activators identified in the primary screen are shown. Transcript levels were normalized to UCP1 levels in ADSCs, n=3 for each technical 20 replicate. *P<0.0.05, **P<0.01, ***P<0.005. FIGS. 8E and 8F are bar graphs showing secondary screening of known thermogenic activators. Transcript levels of UCP1 were assayed in unstimulated ADSCs and ADSC-derived beige adipocytes and following treatment with forskolin (FSK, 20 μM), isoproterenol (1, 10 μM) (8E) and Mirabegron (1, 10 and 25 μM) (8F). Transcript levels were determined by qRT-PCR. n=3 for each independent replicate. Data are mean+/−standard deviation. *P<0.0.05, **P<0.01.

FIGS. 9A-9E are flow cytometric analysis of ADSCs. Approximately 0.5 million human adults ADSCs (ThermoFisher) were immunostained with cell surface markers that characterize mesenchymal cell types (CD105 (9A), CD90 (9B), CD73 (9C), CD45 (9D), CD34 (9E)) for 30 minutes at 4° C. and analyzed by flow cytometry. Matched isotype control for each antibody was used as a negative control. ADSCs used in this study are characterized as CD105+, CD90+, CD73+, CD45- and CD34-. FIG. 9F is a principal component analysis (PCA) plot of ADSCs, compared to mesenchymal stem cells, pre-adipocytes, WAT, macrophage/monocytes, hESCs, endothelial cells and hESC-derived neural stem cells.

FIGS. 10A and 10B are bar graphs showing primary qRT-PCR data comparing effect of growth factors and small molecules on beige adipocyte differentiation and cell survival. Each growth factor or small molecule was added to basal medium with pro-beiging factors (dexamethasone, rosiglitazone, Triiodothyronine and 3-isobutyl-1-methxanthine). IGF1, FGF2 and Y27632 was included with all other factors due to their effect on cell survival. Cells were visually inspected by microscopy and scored, and UCP1 transcript levels were measured by qRT-PCR.

FIG. 11A-11F are bar graphs showing the effect of B8 factors during beige adipogenesis. Each factor was individually removed during the 21 day differentiation to beige adipocytes and differentiation was assessed by qRT-PCR for adipocyte markers (UCP1 (11A), ADIPOQ (11B), PDK4 (11C), PLIN (11D), CIDEA (11E), PPARG (11F)). *P<0.05, **P<0.01, ***P<0.005; bar: 50 μm.

### DETAILED DESCRIPTION OF THE INVENTION

### I. Definitions

As used herein, the term “carrier” or “excipient” refers to an organic or inorganic ingredient, natural or synthetic inactive ingredient in a formulation, with which one or more active ingredients are combined.

As used herein, the term “pharmaceutically acceptable” means a non-toxic material that does not interfere with the effectiveness of the biological activity of the active ingredients.

As used herein, the term “pharmaceutically acceptable carrier” encompasses any of the standard pharmaceutical carriers, such as a phosphate buffered saline solution, water and emulsions such as an oil/water or water/oil emulsion, and various types of wetting agents.

As used herein, the terms “effective amount” or “therapeutically effective amount” means a dosage sufficient to alleviate one or more symptoms of a disorder, disease, or condition being treated, or to otherwise provide a desired pharmacologic and/or physiologic effect. The precise dosage will vary according to a variety of factors such as subject-dependent variables (e.g., age, immune system health, etc.), the disease or disorder being treated, as well as the route of administration and the pharmacokinetics of the agent being administered.

As used herein, the term “prevention” or “preventing” means to administer a composition to a subject or a system at risk for or having a predisposition for one or more symptom caused by a disease or disorder to cause cessation of a particular symptom of the disease or disorder, a reduction or prevention of one or more symptoms of the disease or disorder, a reduction in the severity of the disease or disorder, the complete ablation of the disease or disorder, stabilization or delay of the development or progression of the disease or disorder.

As used herein, the terms “subject,” “individual,” and “patient” refer to any individual who is the target of treatment using the disclosed compositions. The subject can be a vertebrate, for example, a mammal. Thus, the subject can be a human. The subjects can be symptomatic or asymptomatic. The term does not denote a particular age or sex. Thus, adult and newborn subjects, whether male or female, are intended to be covered. A subject can include a control subject or a test subject.

As used herein “comorbidity” means one or more disorders or diseases in addition to the age-related disease or disorder of interest, or an effect of such additional disorders or diseases.

As used herein, the term “gene” refers to a DNA sequence that encodes through its template or messenger RNA a sequence of amino acids characteristic of a specific peptide, polypeptide, or protein. The term “gene” also refers to a DNA sequence that encodes an RNA product. The term gene as used herein with reference to genomic DNA includes intervening, non-coding regions as well as regulatory regions and can include 5′ and 3′ ends.

As used herein, the term “vector” refers to a replicon, such as a plasmid, phage, or cosmid, into which another DNA segment may be inserted so as to bring about the replication of the inserted segment. The vectors can be expression vectors.

As used herein, the term “expression vector” refers to a vector that includes one or more expression control sequences.

As used herein, the term “expression control sequence” refers to a DNA sequence that controls and regulates the transcription and/or translation of another DNA sequence. Control sequences that are suitable for prokaryotes, for example, include a promoter, optionally an operator sequence, a ribosome binding site, and the like. Eukaryotic cells are known to utilize promoters, polyadenylation signals, and enhancers.

As used herein, the terms “transformed,” “transgenic,” “transfected” and “recombinant” refer to a host organism into which a heterologous nucleic acid molecule has been introduced. The nucleic acid molecule can be stably integrated into the genome of the host or the nucleic acid molecule can also be present as an extrachromosomal molecule. Such an extrachromosomal molecule can be auto-replicating. Transformed cells, tissues, or plants are understood to encompass not only the end product of a transformation process, but also transgenic progeny thereof. A “non-transformed,” “non-transgenic,” or “non-recombinant” host refers to a wild-type organism, e.g., a bacterium or plant, which does not contain the heterologous nucleic acid molecule.

As used herein, “substantially changed” means a change of at least e.g. 5%, 10%, 15%, 20%, 25%, 30%, 35%, 40%, 45%, 50%, 75%, 100%, or more relative to a control.

As used herein, the term “purified,” “isolated,” and like terms relate to the isolation of a molecule or compound in a form that is substantially free (at least 60% free, preferably 75% free, and most preferably 90% free) from other components normally associated with the molecule or compound in a native environment.

As used herein, the term “treating” includes alleviating the symptoms associated with a specific disorder or condition and/or preventing or eliminating the symptoms.

### II. Compositions

Adipose tissues play important roles in modulating whole-body energy homeostasis and glucose metabolism. White adipose tissues (WAT) store caloric energy in the form of triglycerides and release fatty acids in response to fed/fasted states. In contrast, brown adipose tissue (BAT) dissipates caloric energy by characteristic UCP1-dependent uncoupled thermogenesis (non-shivering thermogenesis; NST) under cold exposure. In its activated state, BAT consumes a significant amount of glucose and fatty acids in support of the active heat generation in abundant mitochondria. As such, the thermogenic activity of BAT lowers the food efficiency and hence holds promise to correct the positive energy balance. Indeed, compelling evidence from rodent models indicates that increasing the volume or activity of BAT leads to lean and healthy phenotypes, even with excessive caloric intake (Harms, et al., Nat Med, 19, 1252-1263 (2013)).

Studies also indicate that active human BAT resembles UCP1±P° ′ thermogenic adipocytes (also known as beige or brite adipocytes) that scatter within WATs in rodent models (Wu, et al., Cell, 150, 366-376 (2012) and Sharp, L. Z., et al., PLoS One, 7, e49452 (2012)). Furthermore, forskolin can mediate alterations in adipose gene expression (Wu, et al., Cell, 150, 366-376 (2012)).

In rodents, beige adipocytes emerge in WAT depots upon chronic cold stress, sustained sympathetic stimulation or some disease conditions such as cancer-induced cachexia (Harms, et al., Nat Med, 19, 1252-1263 (2013)). In healthy human adults, the activation of beige adipocytes in WATs by cold exposure or sympathetic stimulants (e.g., adrenergic receptor agonists) increases energy expenditure, glucose tolerance, and insulin sensitivity, which indicates the therapeutic potential of beige adipocytes for obesity and diabetes (Saito, et al., Diabetes, 58, 1526-1531 (2009), Wu, et al., Genes & Development, 27:234-250 (2013)). However, BAT mass/activity is low in obese and aged humans (Rogers, N. H., et al., Aging Cell, 11, 1074-1083 (2012) and Berry, D. C., et al., Cell Metab, 25, 166-181 (2017)), who are more likely to be potential patients for BAT-based therapies.

Mouse models revealed that beige adipocytes in WATs might develop from two distinct sources. First, prolonged cold exposure induces de novo beige adipogenic differentiation from perivascular white adipose progenitor cells (Berry, D. C., et al., Nat Commun, 7, 10184 (2016); Long, J. Z., et al., Cell Metab, 19, 810-820 (2014); Vishvanath, L., et al., Cell Metab, 23, 350-359 (2016); and Lee, Y. H., et al., Cell Metab, 15, 480-491 (2012)). Without cold stress, these progenitor cells undergo PPARγ-dependent adipogenic differentiation and give rise to white adipocytes during embryonic and postnatal development. After prolonged cold stress, brown/beige-specific lineage determinants (e.g., Ebf2, Prdm16, PGC1α/β transcription factors/cofactors) activate a thermogenic gene program (e.g., UCP1, ElOVL3, DIO2) in some of the white adipose progenitor cells, which leads to their differentiation into beige adipocytes (Inagaki, T., et al., Nat Rev Mol Cell Biol, 17, 480-495 (2016)). Human beige adipocytes have been shown to develop from capillary networks within cultured human adipose tissue explants, supporting the above lineage determination model of human beige adipocyte formation (Min, S. Y., et al., Nat Med, 22, 312-318 (2016)).

An alternative source of beige adipocytes is terminally differentiated white adipocytes. Recent studies showed that some white adipocytes in mouse models are capable of acquiring the beige adipocyte-specific thermogenesis capacity via a transdifferentiation-like mechanism (white-to-beige adipocyte conversion) upon cold stress or sympathetic simulation (Cinti, S. J Endocrinol Invest, 25, 823-835 (2002); Lee, Y. H., et al., FASEB J, 29, 286-299 (2015); and Rosenwald, M., et al., Nat Cell Biol, 15, 659-667 (2013)). PGC1α/β-dependent mitochondrial biogenesis likely plays an important role in the transformation of mitochondria-less white adipocytes into mitochondria-abundant beige adipocytes (Ikeda, K., et al., Trends Endocrinol Metab, 29, 191-200 (2018)). However, the molecular mechanisms underlying this trans-differentiation type of beige adipocyte formation have not been defined and distinguished with the above-mentioned beige lineage determination mechanisms.

Adipose-derived stem cells (ADSC) (also referred to as Adipose-derived Stem/Stromal Cells (ASCs); Adipose Derived Adult Stem (ADAS) Cells, Adipose Derived Adult Stromal Cells, Adipose Derived Stromal Cells (ADSC), Adipose Stromal Cells (ASC), Adipose Mesenchymal Stem Cells (AdMSC), Lipoblast, Pericyte, Pre-Adipocyte, Processed Lipoaspirate (PLA) Cells) are a plastic adherent cell population isolated from collagenase digests of adipose tissue, including WAT deposits (Bunnell, et al., Methods, 45(2): 115-120 (2008)). ADSC are pluripotent mesenchymal stem cells that are similar to bone marrow-derived mesenchymal stem cells (Sterodimas et al., Aesthet Surg J., 31:682-93 (2011); Tiryaki et al., Aesthet Plast Surg., 965-71 (2011); Park et al., Dermatol Surg., 34:1323-6 (2008)). Indeed, ADSCs are able to differentiate into mature adipose cells, including white adipose tissue, or other adult mesenchymal cells after paracrine or autocrine hormonal stimulations (Yang et al., Expert Opin Biol Ther., 10:495-503, (2010); Takeda et al., Ann Plast Surg., 74:728-36 (2015); Salibian et al., et al., Arch Plast Surg., 40:666-75 (2013)).

Disclosed herein are compositions and methods for the generation of thermogenic adipocytes from ADSC and WAT. Also provided are non-naturally occurring thermogenic adipocytes formed from the disclosed compositions, and methods of distinguishing these thermogenic adipocytes from BAT and beige cells. The desired non-naturally can be formed with high efficiency and purity, in a fully-defined, serum-free medium disclosed herein.

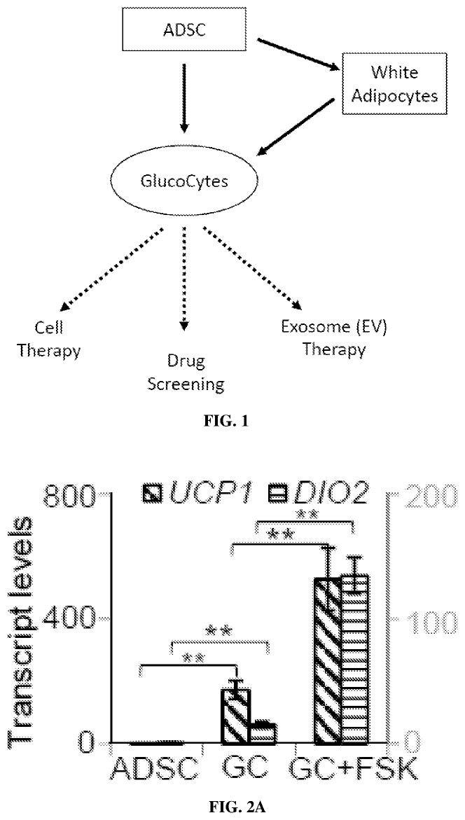

Cells produced by this method exhibit numerous functional characteristics desirable in thermogenic adipocytes including, for example, uncoupled respiration in vitro. Following transplantation into mouse models, the cells show robust glucose uptake, thermogenic activity and contribute to reduced body weight and hyperglycemia. The disclosed thermogenic adipocytes are a therapeutic cell source for treatment of metabolic diseases, and can serve as an in vitro platform drug discovery and drug modelling (FIG. 1).

A. Thermogenic Adipocytes

In vitro and ex vivo derived non-naturally occurring thermogenic adipocytes are provided. In the most preferred embodiments, the ex vivo or in vitro derived thermogenic adipocytes are a unique thermogenic cell type that can be distinguished from other thermogenic cells previously developed in vitro, or that exists in vivo. The cells typically share similar, but not identical, global RNA signatures with other thermogenic adipocytes such as BAT or beige cells, and more so with these thermogenic cells than other adipose cell types such as ADSC and white adipocytes. In a particular embodiment, the non-naturally occurring thermogenic adipocytes are the cells exemplified in the working examples, also referred to herein as GlucoCytes (GC) and beige adipocytes (e.g., ADSC-derived beige adipocytes, etc).

1. Thermogenic Signature

The disclosed non-naturally occurring thermogenic adipocytes typically exhibit a thermogenic molecular signature that can be distinguished from ADSC and white adipocytes. For example, the non-naturally occurring thermogenic adipocytes phenotypically and genetically resemble thermogenic adipocytes in vivo, and can have or exhibit multilocular lipid droplet formation, high mitochondrial content, thermogenic markers, such as UCP1 and/or DIO2, or a combination thereof. Preferably, similar to brown adipose cells, non-naturally occurring thermogenic adipocytes can be activated by forskolin (FSK).

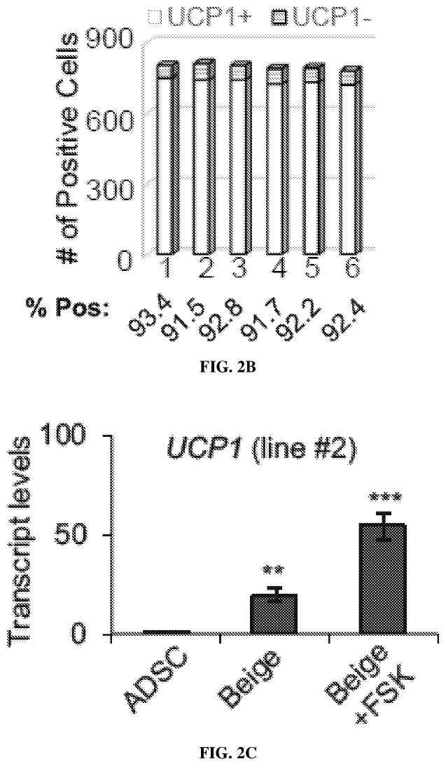

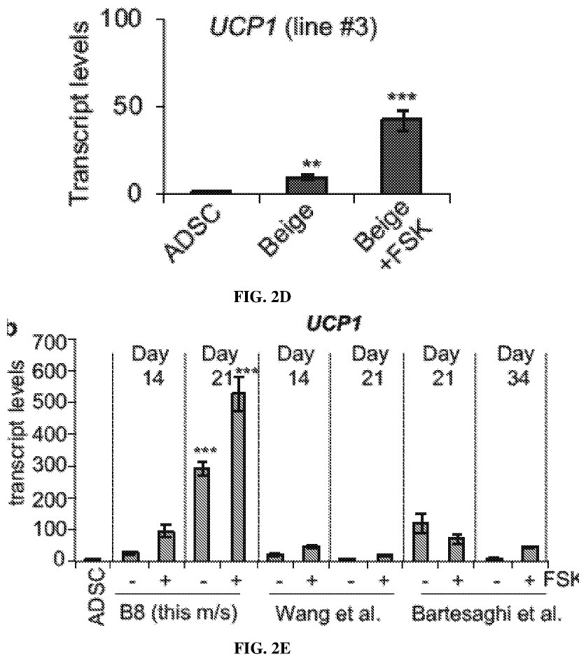

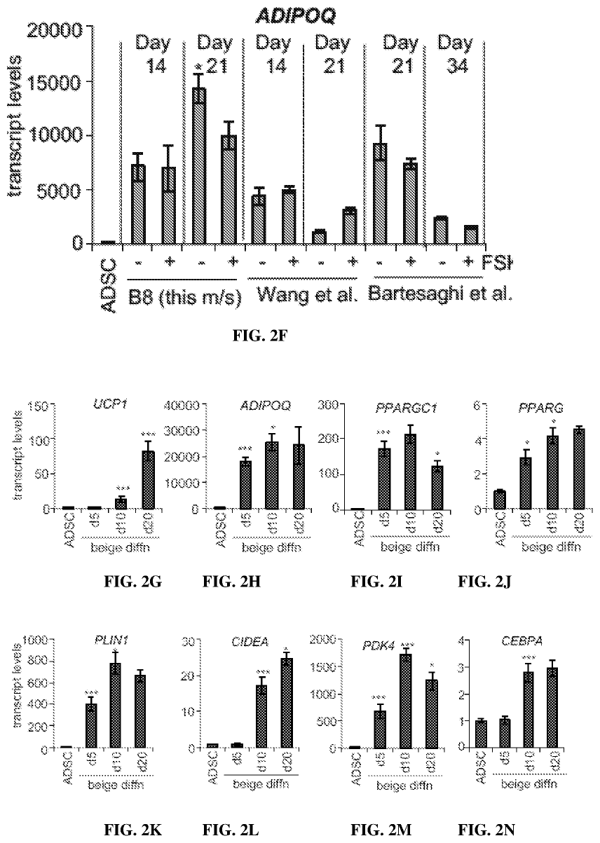

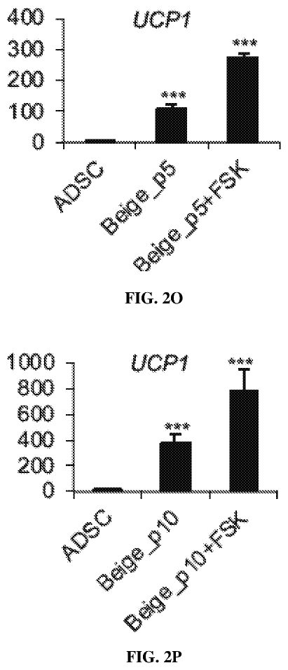

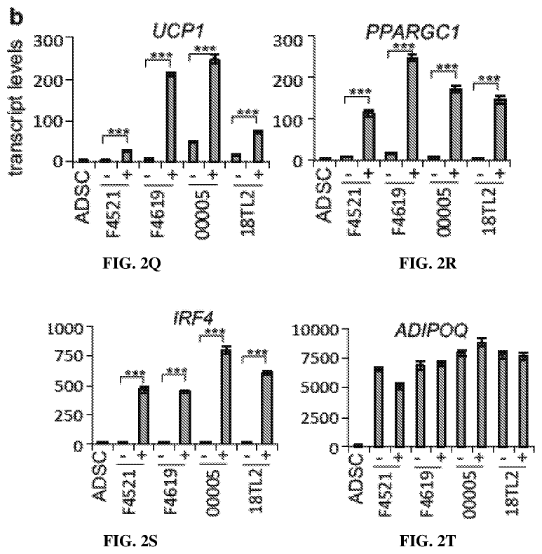

Non-naturally occurring thermogenic adipocytes typically show elevated mRNA and protein expression of uncoupling protein 1 (UCP1), also referred to as thermogenin, relative to, for example, ADSC. UCP1 is an uncoupling protein found in the mitochondria of brown adipose tissue (BAT) and aids in the generations of heat by non-shivering thermogenesis. FIG. 2A-2D show that UCP1 mRNA and protein expression is increased in GC relative to ADSC. Cytologically, the protein can also co-localize with mitochondria and uniformly surround multilocular lipid droplets.

Non-naturally occurring thermogenic adipocytes can also show elevated mRNA expression of the enzyme Type II iodothyronine deiodinase (DIO2) also referred to as iodothyronine 5′-deiodinase, iodothyronine 5′-monodeiodinase, relative to, for example, ADSC. FIG. 2A shows that DIO2 mRNA is increased in GC relative to ADSC.

Activation of non-naturally occurring thermogenic adipocytes including GC by forskolin, may also increase UCP1 and/or DIO2 expression levels relative to untreated cells. See, e.g., FIGS. 2A, and 2C-2D.

Thus, in some embodiments, non-naturally occurring thermogenic adipocytes such as GC can be distinguished from ADSC and/or white adipose cells by having higher UCP1 mRNA and/or protein expression (i.e., UCP1^{high}) higher DIO2 mRNA and/or protein expression (i.e., DIO2^{high}), or a combination thereof, and/or by exhibiting an increase in UCP1 and/or DIO2 following forskolin treatment.

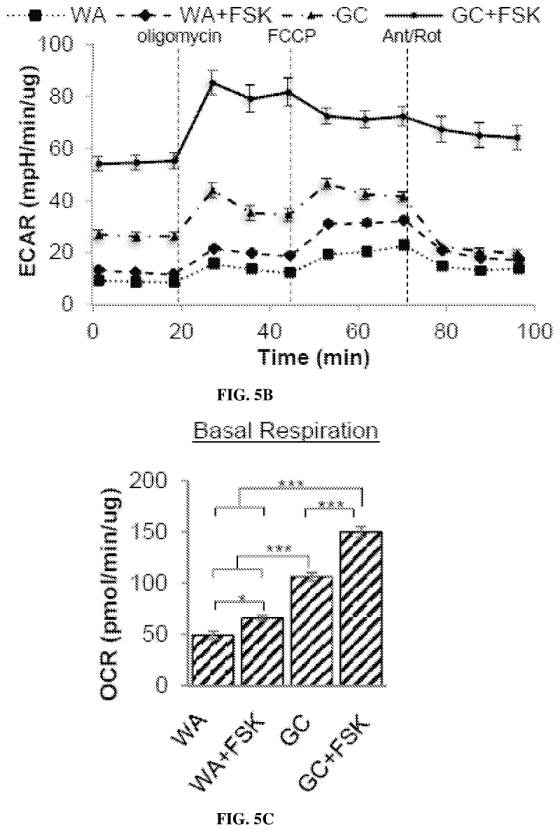

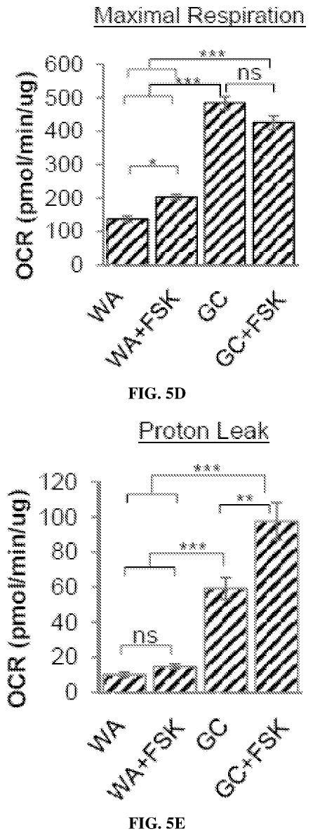

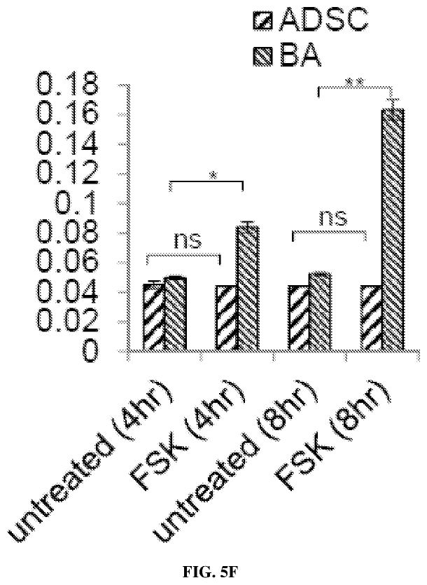

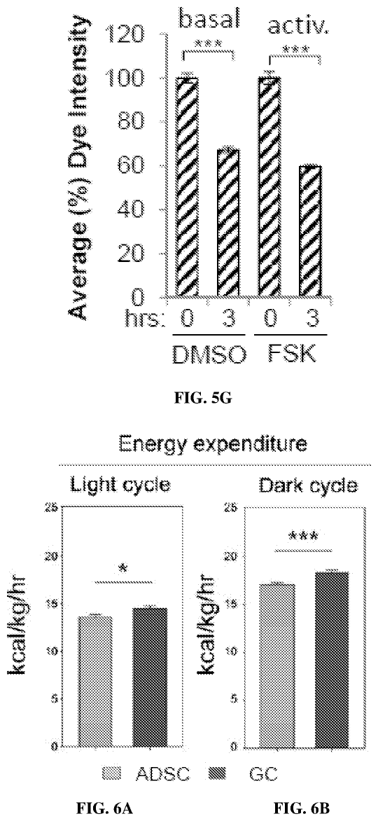

Non-naturally occurring thermogenic adipocytes including GC can also be functionally distinguished from white adipose (WA) cells. For example, non-naturally occurring thermogenic adipocytes including GC can have higher levels of mitochondrial respiration, glycolysis, or a combination thereof relative to, for example, WA. In some embodiments, this difference can be increased by treatment with forskolin (see e.g., FIGS. 5A and 5B). For example, basal and/or maximal respiration can be elevated in the non-naturally occurring thermogenic adipocytes compared to white adipocytes. See, e.g., FIGS. 5C and 5D. In some embodiments, uncoupled respiration (proton leak) is increased in non-naturally occurring thermogenic adipocytes including GC compared to the WA, and further elevated upon FSK treatment. See, e.g., FIG. 5E. Thus, non-naturally occurring thermogenic adipocytes including GC are metabolically distinct from WA in vitro and have a metabolic capacity for uncoupled respiration similar to other thermogenic adipocytes in vivo.

Thermogenic adipocytes, upon stimulation with forskolin, are known to induce lipolysis and activate their thermogenic programs. In some embodiments, non-naturally occurring thermogenic adipocytes including GC exhibit evidence of lipolysis and/or activation of a thermogenic program. For example, in some embodiments, free glycerol is increased in the non-naturally occurring thermogenic adipocytes' microenvironment (e.g., in response FSK treatment) indicating increased lipolysis. A reduction in thermogenic dye (e.g., ERthermAC) signal intensity (e.g., in response to FSK treatment) can indicate an increase in thermogenic adipocyte activation (e.g., thermogenesis). See, e.g., FIGS. 5F and 5G.

In some embodiments, non-naturally occurring thermogenic adipocytes including GC elevate energy expenditure and/or oxygen consumption in a subject following transplantation therein relative to ADSC, and preferably without a change in the respiratory exchange ratio and/or locomotor activity in the subject. In some embodiments, non-naturally occurring thermogenic adipocytes transplantation increases core body temperature, induces weight loss or reduces weight gain, or a combination thereof, preferably without significantly changing water consumption, hydration, food intake, and/or muscle mass in the subject.

2. Distinguishable from Other Thermogenic Cells

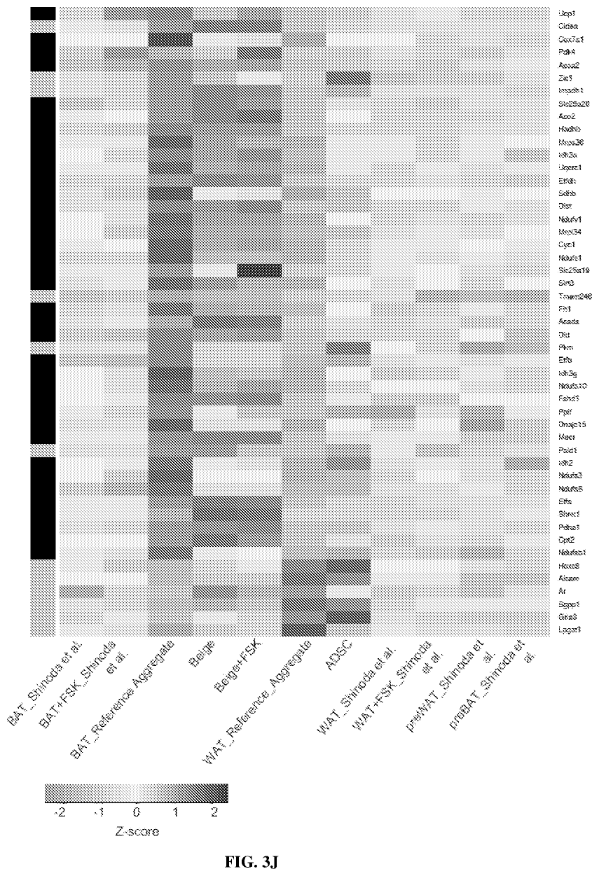

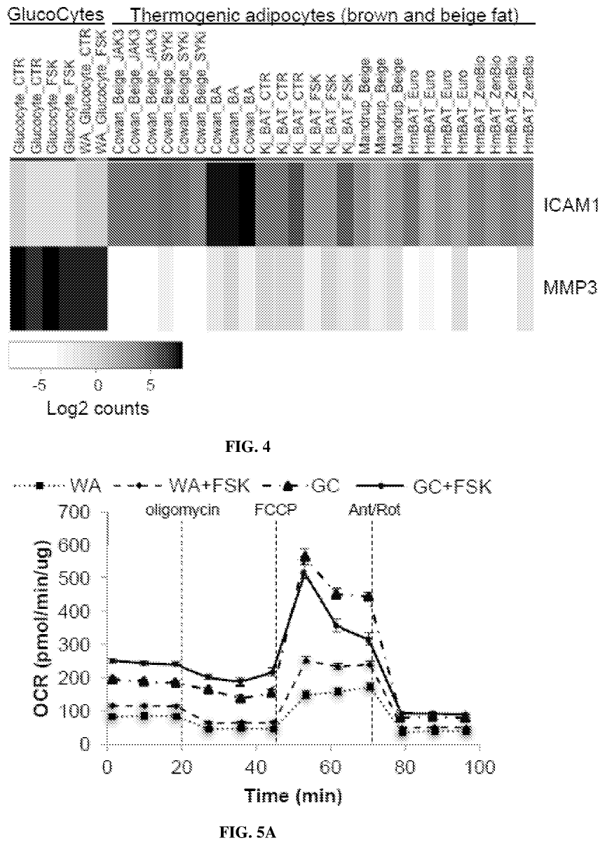

Non-naturally occurring thermogenic adipocytes such as GC can also exhibit a unique molecular signature that distinguishes them from other naturally occurring and induced thermogenic cells. See, e.g., FIG. 4, which shows a heatmap comparing markers for GC and other thermogenic adipocytes from in vitro or in vivo sources by RNA-sequencing.

For example, mRNA and protein expression studies show that GC express lower levels of the surface glycoprotein Intercellular Adhesion Molecule 1 (ICAM1) also known as CD54 (Cluster of Differentiation 54), relative to other thermogenic adipocytes including both BAT and beige cells. mRNA and protein expression studies also show that GC express higher levels of the enzyme matrix metalloproteinase-3 (MMP3), also known as stromelysin-1, relative to other thermogenic adipocytes including both BAT and beige cells Immunostaining confirmed the expression patterns for MMP3 and ICAM-1 in GlucoCytes.

Thus, in some embodiments, non-naturally occurring thermogenic adipocytes including GC can be distinguished from other closely related cell types such as BAT and beige cells by having lower ICAM1 mRNA and/or protein expression (i.e., ICAM1^{low}), higher MMP3 mRNA and/or protein expression (i.e., MMP3^{high}), or a combination thereof.

B. Thermogenic Adipocyte Differentiation Medium

Culture medium (also referred to media) for in vitro and ex vivo differentiation of adipose tissue-derived stem cells (ADSCs) and/or white adipose cells into thermogenic adipocytes, and preferably GC, is provided.

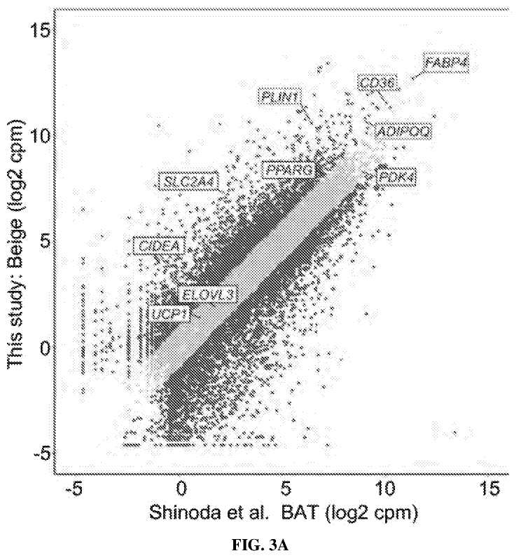

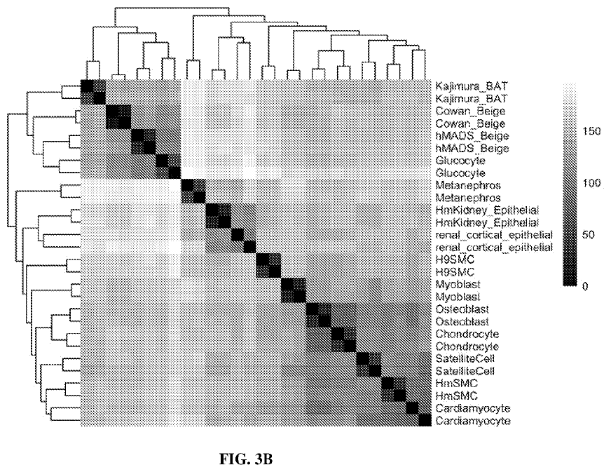

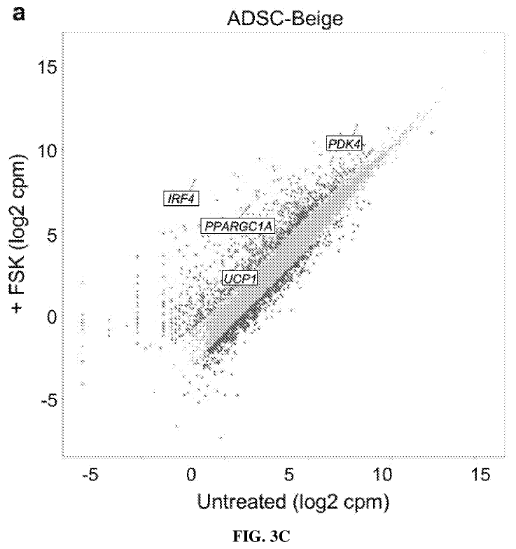

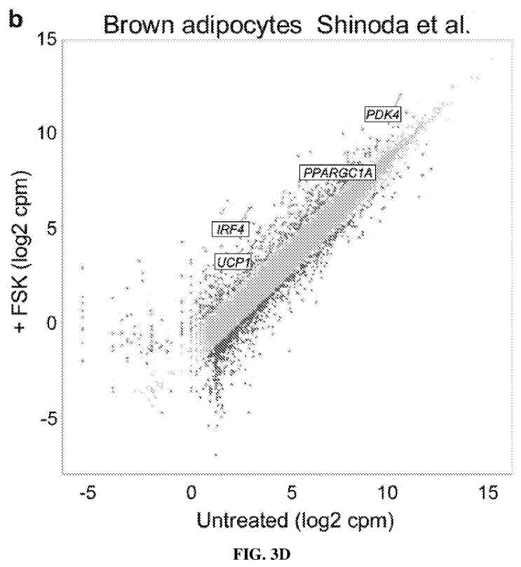

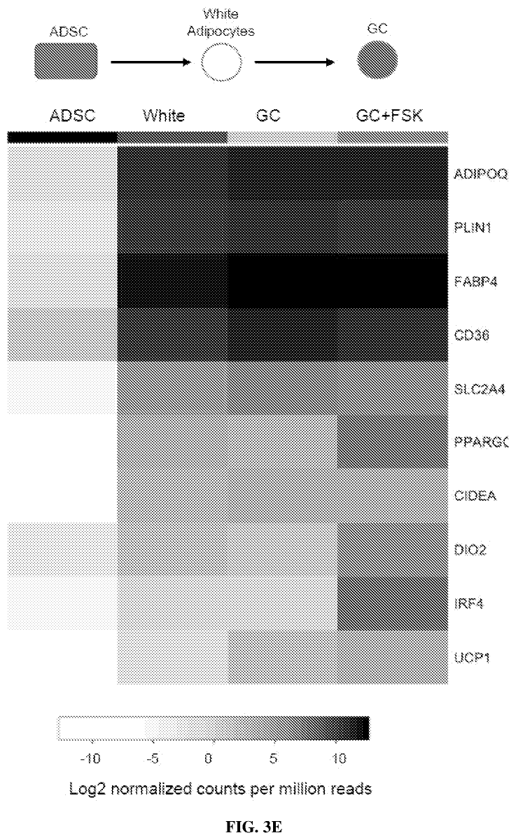

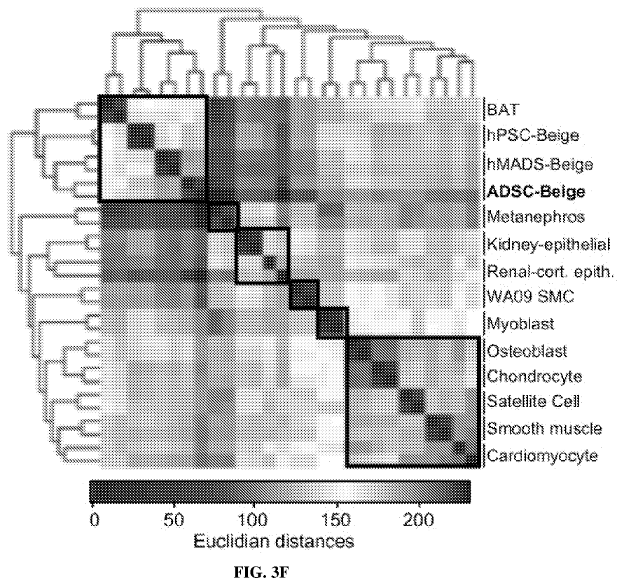

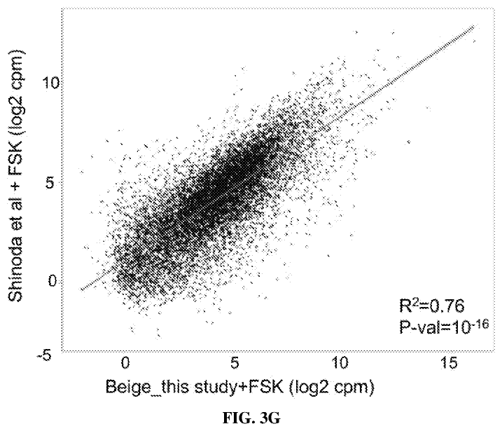

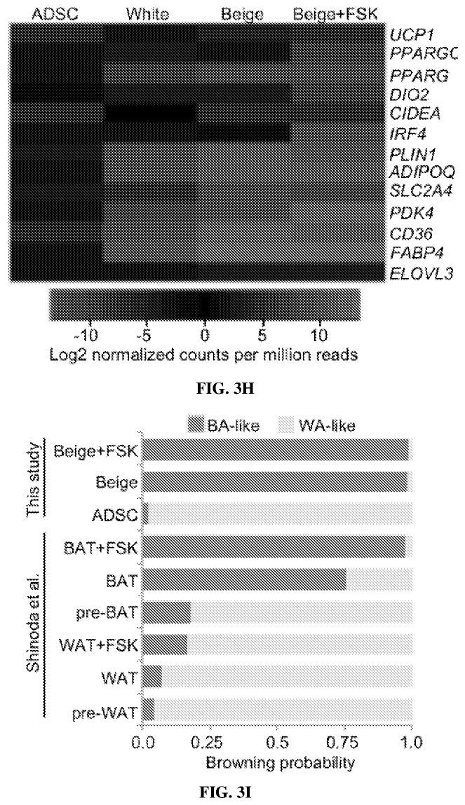

However, as introduced above, and exemplified below, the disclosed compositions and methods induce differentiation of ADSCs into thermogenic adipocytes such that the resulting cells are structurally and/or functionally distinct with other cells generated in vitro, ex vivo, or naturally occurring in vivo. The Examples also show that GC can be formed by differentiating white adipocytes (see, e.g., FIG. 1 and FIG. 3E), including, but not limited to, white adipocytes formed from differentiating ADSC.

Differentiation medium typically includes one or more of eight growth factors and/or inhibitors preferably in combination with one or more additives relative to a base tissue culture media. Base media, exemplary additives that can be used to form chemically defined medium, exemplary growth factors and inhibitors that can be used to form differentiation medium, as well as preferred concentrations thereof are discussed in more detail below.

1. Chemically Defined Medium

The base medium is a tissue culture medium, for example a commercially available tissue culture medium. The culture medium is typically a synthetic culture medium, which is composed of balanced salt solution(s), and amino acids and vitamins A preferred base medium is DMEM/F-12. Other exemplary base mediums include, for example, EMEM, DMEM, F-12, IMDM, and RPMI. In some embodiments, the base medium initials lack glutamine. However, as discussed in more detail below, the medium can be supplemented with a glutamine additive, for example, Corning® Glutagro™ Supplement.

The base medium is typically supplemented with one or more additives to form a chemically defined medium (DM). Preferred additives include, but are not limited to, bovine serum albumin (BSA) (e.g., Probumin®), one or more antibiotics and/or antimycotics (e.g., Gibco® Antibiotic-Antimycotic solution which includes penicillin, streptomycin, and Gibco® Amphotericin B), one or more non-essential amino acids (e.g., L-alanine, L-asparagine, L-aspartic acid, L-glycine, L-serine, L-proline and L-glutamic acid), one or more components of Trace Elements A (e.g., cupric sulfate, ferric citrate, sodium selenite, zinc sulfate, and combinations thereof), one or more components of Trace Elements B (e.g., ammonium molybdate, ammonium vanadate, manganese sulfate, nickel sulfate, sodium silicate, stannous chloride, hydrochloric acid, and combinations thereof), one or more components of Trace Elements C (e.g., aluminum chloride, barium acetate, cadmium chloride, chromic chloride, cobalt dichloride, germanium dioxide, potassium bromide, potassium iodide, rubidium chloride, silver nitrate, sodium fluoride, zirconyl chloride, and combinations thereof), ascorbic acid, transferrin, 2-mercaptoethanol, and/or L-glutamine e.g., a stabilized dipeptide form thereof such as Corning® Glutagro™ Supplement.

In some embodiments, the chemically defined medium includes one or more of the following factors in Table 1 added to a base medium such as DMEM/F-12.

TABLE 1
Exemplary Factors for Chemically Defined Medium

| | Final |
|---|---|
| Compound | Conc. |
| bovine serum albumin (BSA) (e.g., Probumin ®) | 2% |
| one or more antibiotics and/or antimycotics (e.g., Gibco ® Antibiotic- | 1X |
| Antimycotic solution which includes penicillin, streptomycin, and Gibco | |
| Amphotericin B) | |
| one or more non-essential amino acids (e.g., L-alanine, L-asparagine, L- | 1X |
| aspartic acid, L-glycine, L-serine, L-proline and L-glutamic acid) | |
| L-glutamine e.g., a stabilized dipeptide form thereof such as Corning ® | 1X |
| glutagro ™ Supplement | |
| one or more components of Trace Elements A (e.g., cupric sulfate, ferric | 1X |
| citrate, sodium selenite, zinc sulfate, and combinations thereof) | |
| one or more components of Trace Elements B (e.g., ammonium | 1X |
| molybdate, ammonium vanadate, manganese sulfate, nickel sulfate, | |
| sodium silicate, stannous chloride, hydrochloric acid, and combinations | |
| thereof) | |
| one or more components of Trace Elements C (e.g., aluminum chloride, | 1X |
| barium acetate, cadmium chloride, chromic chloride, cobalt dichloride, | |
| germanium dioxide, potassium bromide, potassium iodide, rubidium | |
| chloride, silver nitrate, sodium fluoride, zirconyl chloride, and | |
| combinations thereof) | |
| L-ascorbic acid | 50 μg/ml |
| Transferrin | 10 μg/ml |

2. Growth Factors and Inhibitors

The disclosed differentiation medium typically includes one or more growth factors, one or more inhibitors, or a combination thereof. The growth factor(s) and/or inhibitor(s) can be added to the base medium or chemically defined medium. For example, the differentiation medium can include one or more insulin-like growth factors, fibroblast growth factors, bone morphogenetic proteins, ROCK family of kinase inhibitors, insulin sensitizers, thyroid hormones, glucocorticosteroids, cyclic nucleotide phosphodiesterase inhibitors, or a combination thereof. In some embodiments, the medium includes one or more from 2, 3, 4, 5, 6, 7, or all 8 of the foregoing classes of the growth factors and/or inhibitors. For example, the differentiation medium can include at least one each of an insulin-like growth factor, a fibroblast growth factor, a bone morphogenetic protein, a ROCK family of kinase inhibitor, an insulin sensitizer, a thyroid hormone, a glucocorticosteroid, and a cyclic nucleotide phosphodiesterase inhibitor.

In preferred embodiments, the insulin-like growth factor is insulin-like growth factor 1 (IGF-I), the fibroblast grow factor is basic fibroblast growth factor (bFGF or basic-FGF), the bone morphogenetic protein is bone morphogenetic protein 7 (BMP7), the ROCK kinase inhibitor is Y27632, the insulin sensitizer is rosiglitazone, the thyroid hormone is triiodo-L-thyronine, the glucocorticosteriod is dexamethasone, and the cyclic nucleotide phosphodiesterase inhibitor is isobutylmethylxanthine (IBMX). In preferred embodiments, the insulin-like growth factor, fibroblast growth factor, and/or bone morphogenetic protein are human growth factors.

In some embodiments, the medium includes 1, 2, 3, 4, 5, 6, 7, or all 8 of the following growth factors and/or inhibitors in the concentrations in Table 2.

TABLE 2
Exemplary Growth Factors and Inhibitors and Concentrations

| Compound | Final Conc. | |
|---|---|---|
| BMP7 | 100 | ng/ml |
| IGF-1 | 200 | ng/ml |
| FGF2 | 8 | ng/ml |
| Y-27632 | 10 | μM |
| Rosiglitazone | 2 | μM |
| Dexamethasone | 1 | μM |
| Triiodo-L-thyronine | 1 | nM |
| (LT3) | | |
| IBMX | 500 | μM |

3. Exemplary Differentiation Medium

In a specific embodiment, the differentiation medium is B-8. B-8 medium is defined according to Table 3.

TABLE 3
Composition of B-8 Medium.

| | | Final | |
|---|---|---|---|
| Compound | Diluent | Conc. | |
| Probumin ® | DMEM/F-12 | 2% | |
| Pen/Strep/anti- | — | 1X | |
| mycotic | | | |
| Nonessential amino | — | 1X | |
| acids | | | |
| GlutaGro ™ | — | 1X | |
| Trace Elements A | — | 1X | |
| Trace Elements B | — | 1X | |
| Trace Elements C | — | 1X | |
| L-ascorbic acid | ddH2O | 50 | ug/ml |
| Transferrin | DMEM/F-12 | 10 | ug/ml |
| DMEM/F-12 | — | 1X | |
| BMP7 | 4 mM | 100 | ng/ml |
| | HCL + 0.2% Probumin | | |
| IGF-1 | 4 mM | 200 | ng/ml |
| | HCl + 0.2% BSA | | |
| FGF2 | PBS + 0.2% BSA | 8 | ng/ml |
| Y-27632 | DMSO | 10 | μM |
| Rosiglitazone | DMSO | 2 | μM |
| Dexamethasone | DMSO | 1 | μM |
| Triiodo-L-thyronine | DMSO | 1 | nM |
| (LT3) | | | |
| IBMX | DMSO | 500 | μM |

In particular embodiments, the media include at least dexamethasone, rosiglitazone, Triiodothyronine and 3-isobutyl-1-methxanthine, preferably in combination with at least IGF1, FGF2 and Y27632.

In some embodiments, the media further includes BMP4 (e.g., 50 ng/ml).

In some embodiments, the media includes or excludes any one or more of: all-trans retinoic acid, PD0325901, BIO, CHIR99021, LY294002, Rapamycin, SB431542, insulin, indomethacin, forskolin, ascorbate-2-phosphate, fetal bovine serum, and/or fetal calf serum. See e.g., Tables 5 and 6.

4. Methods of Making Differentiation Medium

Differentiation medium can be made by adding one or more of the growth factors and/or inhibitors preferably in combination with adding one or more additives introduced above to a base medium. Differentiation medium typically includes both growth factors and inhibitors as well as additives relative to the base tissue culture medium. The growth factors and inhibitors can be added before, after, or at the same time as the additives.

For example, a chemically defined medium can be formed by supplementing a base medium such as DMEM/F12 to a final concentration of 2% BSA, 1× antibiotic and/or antimyotic, 1× non-essential amino acids (L-alanine, L-asparagine, L-aspartic acid, L-glycine, L-serine, L-proline and L-glutamic acid), 1× Trace Elements A (Cupric Sulfate, Ferric Citrate, Sodium Selenite, and Zinc Sulfate), 1× Trace Elements B (ammonium molybdate, ammonium vanadate, manganese sulfate, nickel sulfate, sodium silicate, stannous chloride, and hydrochloric acid), 1× Trace Elements C (aluminum chloride, barium acetate, cadmium chloride, chromic chloride, cobalt dichloride, germanium dioxide, potassium bromide, potassium iodide, rubidium chloride, silver nitrate, sodium fluoride, and zirconyl chloride), 50 μg/mL ascorbic acid, 10 μg/mL transferrin, 0.1 mM 2-mercaptoethanol and 1× stabilized dipeptide form of L-glutamine (e.g., Corning® Glutagro™ Supplement).

The based medium or chemically defined medium can be supplemented to a final concentration of 200 ng/mL IGF-I, 8 ng/mL basic-FGF, 100 ng/mL BMP7, 10 μM Y27632, 2 μM rosiglitazone, 1 nM triiodo-L-thyronine, 1 μM dexamethasone, and 500 μM isobutylmethylxanthine.

Typically, the differentiation medium is serum-free.

C. Conditioned Medium

Also provided are materials secreted by non-naturally occurring thermogenic adipocytes including GCs. Exemplary materials include, but are not limited to, secreted factors and extracellular vesicles. The secreted factors can be cytokines and other cell signing proteins secreted by adipose tissue, also referred to as adipokines. Exemplary secreted factors include, but are not limited to, leptin, adiponectin, apelin, chemerin, interleukin-6 (IL-6), monocyte chemotactic protein-1 (MCP-1), plasminogen activator inhibitor-1 (PAI-1), retinol binding protein 4 (RBP4), tumor necrosis factor-alpha (TNFα), visfatin, omentin, vaspin, progranulin, CTRP-4, interleukin 8 (IL-8), interleukin 10 (IL-10), interferon gamma (IFN-γ) and inducible protein 10 (IP-10 or CXCL10) have been shown to be associated with excessive body weight.

Other secreted factors include, for example, secreted RNAs such as miRNAs. In some cases, these may be within exosomes. In other cases these may be attached to secreted proteins (see, e.g., Chen, et al., Trends Cell Biol., 22(3):125-32 (2012) doi: 10.1016/j.tcb.2011.12.001).

The extracellular vesicles can include, for example, exosomes and/or microvesicles.

The secreted materials can be collected using standard methods are well known in the art. Standard methods typically include washing the cells and adding serum-free medium, allowing the cells to condition the medium for a desired period of time, typically about 1 to about 3 days, and collecting the conditioned medium.

The conditioned medium can be utilized neat, or can be further processed to increase the concentration of desired materials, and/or separate some materials from others.

For example, centrifugation can be used to clear the media of cells. Higher speed, and preferably density centrifugation. See, e.g., Witwer, et al., J Extracell Vesicles, 2: 10.3402/jev.v2i0.20360 (2013). Proteins can be precipitated by using, for example, ammonium sulfate, and collected by, for example centrifugation.

### III. Methods of Making Thermogenic Adipocytes

Methods of making thermogenic adipocytes including GlucoCytes are also provided. The methods typically include differentiating ADSCs or white adipose cells into thermogenic adipocytes, preferably GCs. The methods can also include isolation and/or expansion of ADSCs or white adipose cells.

A. Methods of Isolating ADSCs and WAT

Source of ADSC and WAT include cell lines, donor materials, and freshly harvested cells. For example, the technology is used for personalized therapy. Target cells can be first isolated from a donor using methods known in the art, differentiated in vitro (ex vivo), and administered to a patient in need thereof. In some embodiments, the target cells to be administered to a subject will be autologous, e.g. derived from the subject, or syngeneic. Allogeneic cells can also be isolated from antigenically matched, genetically unrelated donors (identified through a national registry), or by using target cells obtained or derived from a genetically related sibling or parent.

The methods can include the isolation and or preparation of ADSCs or white adipocytes. ADSCs can be isolated from a subject. For example, in some embodiments, ADSCs are isolated from fat aspirates. Methods of isolating ADSCs, such as from fat aspirates, are known in the art. See, for example, Sterodimas, et al., Aesthet Surg J., 31:682-93 (2011), Trivisonno, et al., Aesthetic Surgery Journal, 34(4) 601-613 (2014), Zhu, et al., J Vis Exp., (79):50585, doi:10.3791/50585 (2013), Wankhade, et al., Stem Cells Int., 2016: 3206807, 9 pages (2016) and Savoia, et al., Biomed Dermatol, 1: 1. doi.org/10.1186/541702-017-0003-6 (2017).

For example, adipose tissue can be extracted from a subject. In some embodiments the extraction is by liposuction or a variation thereon, which allows for collection of large amounts adipose tissue with minor donor site morbidity (Yoshimura, et al., J Cell Physiol., 208:64-76. doi: 10.1002/jcp.20636 (2006)). Typical isolation procedures for ADSCs can include mechanical dissociation and/or enzymatic collagenase digestion of the lipoaspirated tissue followed by centrifugation to obtain the component cellular fractions, which generates a high density of stromal-vascular fraction (SVF) (Moustaki, et al., Exp Ther Med., 14(3): 2415-2423 doi: 10.3892/etm.2017.4811 (2017)). The SVF contains a highly heterogeneous cell population, including endothelial cells, endothelial progenitor cells (EPC), pericytes, preadipocytes, fibroblasts, blood-derived cells and vascular smooth muscle cells, in addition to the potential ADSCs (Yoshimura, et al., J Cell Physiol., 208:64-76. doi: 10.1002/jcp.20636 (2006)).

ADSCs are present as a minor fraction within the SVF. SVF may be used directly as a source of ADSCs, or ADSCs can be separated from SVF and expanded in culture by adhesion on plastic dishes (Yoshimura, et al., Aesthet Plast Surg., 32:48-57. doi: 10.1007/s00266-007-9019-4 (2008), Fraser, et al., Cytotherapy, 9:459-467. doi: 10.1080/14653240701358460 (2007)). The isolation of ADSCs from the SVF is a simple, uncomplicated in vitro procedure. In general, cell isolation protocols include collagenase-digestion of lipoaspirate or minced adipose tissue followed by density gradient centrifugation of the enzyme-digested tissue. Stem cell yields are greater from adipose tissue than from other stem cell sources (Zhu, et al., Cell Biochem Funct., 26:664-675. doi: 10.1002/cbf.1488 (2008)). As many as 1×10⁷ ADSCs may routinely be isolated from 300 ml of lipoaspirate, following ex vivo culture, with a yield of 5,000 fibroblast colony-forming units per g of adipose tissue, which is 5× the estimate of the ADSC yield from bone marrow (Sterm, et al., Keio J Med., 54:132-141, doi: 10.2302/kjm.54.132 (2005), Boquest, et al., Methods Mol Biol., 325:35-46 (2006)).

White adipocytes can obtained from WAT compartments in a subject, including, for example, superficial subcutaneous, deep subcutaneous, omental and mesenteric WAT. WAT tissue can be differentiated into thermogenic adipocytes including GC, or serve as a source for ADSC. ADSC can be differentiated into thermogenic adipocytes including GC, or be differentiated into WAT, which in turn can be differentiated into thermogenic adipocytes including GC.

B. Methods of ADSC Expansion

The ADSCs can be cultured and/or expanded in vitro or ex vivo prior to differentiation. For example, in the examples below, ADSCs (for example, at a density of about 5,000 cell/cm²) were seeded and cultured prior to differentiation.

ADSCs can be cultured in ADSC-growth medium. ADSCs can be passaged and expanded for several months, usually at least 10 passages (performed weekly) until they senesce. An exemplary ADSC-growth medium utilized to culture ADSC in the experiments below includes 10% fetal bovine serum in DMEM media with 1× Antibiotic-Antimycotic, 1× MEM non-essential amino acids, 1× Glutagro™ and 1×2-Mercaptoethanol (BME). The medium can be changed as-needed, for example every 1, 2, 3, 4, or 5 days. In a particular embodiment, the medium is changed every other day.

The cells can be cultured for, e.g., at least 2 days, at least 5 days, at least 10 days, at least 15 days, etc. In particular embodiments, the ADSCs are grown in ADSC-growth medium for 1-10 days, or 2-8 days, or 3-7 days, or 4-6 days, or about 5 days, or 5 days, or any specific number or range of days between 1-10 days. Typically, the cells are passaged when they become at least 50%, 60%, 70%, 75%, 80%, 95%, 90%, or 95% confluent. In particular embodiments, the cells are cultured to about 75%-95%, or preferably about 80%-90%, confluent before passaging.

In the experiments below, ADSCs were expanded by seeding cells at a density of about 5,000 cell/cm² on 100 mm cell culture plates and passaging them at 80-90% confluency, which took approximately 5 days. To passage cells from a 100 mm cell culture plate, ADSCs were first washed with DPBS and then incubated with 5 ml of Accutase for 5 minutes at room temperature. Next, 5 ml of DPBS was added and cells were centrifuged at 1000 rpm for 4 min in a swinging bucket centrifuge. Cells were resuspended in ADSC-growth medium and counted using a hemocytometer for seeding. Following cell seeding, media was changed every other day. Such methods can also be adjusted for use with larger or small tissue culture plates.

C. Methods of Differentiation

Methods of differentiating ADSC and white adipocytes into thermogenic adipocytes such as GC typically include culturing ADSC or WAT cells, preferably confluent cells, for one or more days in differentiation medium such as B-8 until the cells differentiate into thermogenic adipocytes, preferably GC. In some embodiments, the cells are cultured for about 1 to 100 days, or about 5 to 75 days, or about 10 to 50 days, or about 15 to 25, or about 18-23 days or about 21 days, or any other specific number of days or range of days between 1 and 100. Exemplary GC differentiation assays are described below and were used to generate the GC utilized in the experiments described herein. The ADSC in the experiments were cultured in B-8 medium for three weeks to generate GC. Thus, in preferred embodiments, the ADSC or white adipocytes are cultured in B-8 medium, preferably for about three weeks. The media can be changed as-needed, for example every 1, 2, 3, 4, or 5 days. In a particular embodiment, the media is changed every other day.

In some embodiments, ADSC are first differentiated into WAT and subsequently differentiated into thermogenic adipocytes such as GC. To perform white adipocyte differentiations, ADSCs can be passaged and seeded at a density of, for example, 5000 cell/cm², grown to confluency, and differentiated using the StemPro adipogenesis differentiation kit (ThermoFisher, A10070-01). Media can be changed, for example, every two days for 3 weeks.

Any of the disclosed tissue culturing methods can include standard tissue practices including, but not limited to, passaging the cells, changing the medium, etc. The cells are typically incubated in standard tissue culture conditions (e.g., Temperature at 37° C.; CO2 at 5%, Relative Humidity as 95%

### IV. Methods of Use

A. Methods of Treatment

The disclosed methods of treatment typically includes administering a subject in need thereof an effective amount of a composition such as, or including, non-naturally occurring thermogenic adipocytes such as GC, a composition derived therefrom such as secreted factor(s), an active agent identified as a modulator of adipocytes such as themogenic adipocytes, or a combination thereof.

In some embodiments, the effect of the disclosed compositions and methods on a subject is compared to a control. For example, the effect of the composition on a particular symptom, pharmacologic, or physiologic indicator can be compared to an untreated subject, or the condition of the subject prior to treatment. In some embodiments, the symptom, pharmacologic, or physiologic indicator is measured in a subject prior to treatment, and again one or more times after treatment is initiated. In some embodiments, the control is a reference level, or average determined based on measuring the symptom, pharmacologic, or physiologic indicator in one or more subjects that do not have the disease or condition to be treated (e.g., healthy subjects). In some embodiments, the effect of the treatment is compared to a conventional treatment that is known the art, such as one of those discussed herein.

In some embodiments, the compositions and methods are effective to reduce one or more symptoms of a disease, disorder, and condition to be treated. Such diseases, disorders, and conditions and more specific symptoms thereof are discussed in more detail below.

For example, in some embodiments, the cells or secreted factor(s) or active agents are administered to the subject in an effective amount to, for example, increase glucose uptake, increase thermogenic activity, reduce weight gain or reduce body weight (e.g., increase weight loss), increase energy expenditure, reduce hyperglycemia, increase VO₂, increase energy expenditure, increase core temperature, or a combination thereof. In some embodiments, the cells, secreted factor(s), or active agent improve insulin resistance (e.g., reduce insulin resistance), improve glucose tolerance, improve insulin sensitivity, or a combination thereof in a subject. In some embodiments, respiration exchange ratio and/or activity levels are not significantly changed in treated subjects.

1. Cell Transplantations

In some embodiments, the methods include administering a subject in need thereof an effective amount of thermogenic adipocytes such as GC. Methods of transplanting adipose cells for therapeutic purposes are discussed in, for example, Trans and Khan, Nat Rev Endocrinol., 6(4): 195-213 (2010), which is specifically incorporated by reference herein in its entirety. Briefly, cells can be delivered by transplantation, subcutaneous injection, site-specific injection, or intravenous injection.

In certain embodiments, the cells are administered locally, for example by injection or other application directly into or onto a site to be treated. In some embodiments, the cells are injected or otherwise administered directly into adipose tissue. Typically, local administration causes an increased localized concentration of the cells which is greater than that which can be achieved by systemic administration (e.g., intravenous injection).

Transplantation of non-naturally occurring thermogenic adipocytes such as GC can employ methods and procedures similar to those for islet cell transplantation. Cells can be administered to one (or more) of several different sites including, but not limited to, liver, kidney subcapsule, spleen, pancreas, peritoneum and omental pouch, gastrointestinal wall, intramuscular and subcutaneous space, bone marrow. See, e.g., Rajab, et al., Curr Diab Rep. 10:332-7 (2010). The number of cells to be transferred can parallel those of established mesenchymal stem cell (MSC) transplantations. See Meier, et al., Stem Cell Research, 11(3): 1348-1364 (2013). For example, in particular embodiments, non-naturally occurring thermogenic adipocytes such as GC are administered to a subject in need thereof at about 1 million cells/kg weight of recipient.

Cell formulations can be prepared using a pharmaceutically acceptable “carrier” composed of materials that are considered safe and effective and may be administered to an individual without causing undesirable biological side effects or unwanted interactions. The “carrier” is all components present in the pharmaceutical formulation other than the cells. The term “carrier” includes, but is not limited to, diluents, binders, lubricants, disintegrators, fillers, and coating compositions. For example, in some embodiments, the cells are delivered in a cell media or physiological salt solution.

The cells can be administered to a subject with or without the aid of a delivery vehicle or depot. Appropriate delivery vehicles and depots for cells are known in the art. For example, increased cell survival and lipid content of ASCs differentiated into fat after transplantation have been reported with the use of hydrogels (Tan, et al., Biomaterials, 30(36):6844-53 (2009)), PLGA (poly(lactic-co-glycolic acid)) (Choi, et al., Biomaterials, 26:5855-5863 (2005)), and collagen scaffolds (Itoi, et al., J Plast. Reconstr. Aesthet. Surg., 63(5):858-64 (2009)).

Any of the methods can further include delivery, preferably local delivery, of one or more factors to enhance angiogenic, antifibrotic, anti-apoptotic and anti-inflammatory properties, such as VEGF (Rehman, et al., Circulation, 109:1292-1298 (2004), Yi, et al., J Plast. Reconstr. Aesthet. Surg., 60:272-278 (2007)) hepatic growth factor (HGF) ((Rehman, et al., Circulation, 109:1292-1298 (2004), Zhu, et al., Biochem. Biophys. Res. Commun., 379:1084-1090 (2009)) fibroblast growth factor (FGF) (Bhang, et al., Stem Cells, 27:1976-1986 (2009)), transforming growth factor (TGF) 13 (Rehman, et al., Circulation, 109:1292-1298 (2004)), platelet-derived growth factor (PDGF) (Craft, et al., J Plast. Reconstr. Aesthet. Surg., 62:235-243 (2009)), IL-8 (Shoshani, et al., Plast. Reconstr. Surg., 115:853-859 (2005)), or matrix metalloproteinase (MMP) 2 (Kuromochi, et al., Eur. J Clin Invest., 38:752-759 (2008)), have been shown to increase survival of fat grafts.

In some embodiments, the cells are additionally programmed to increase, encourage, or otherwise maintain a thermogenic phenotype in the cells (e.g., increased expression of UCP1, PPARγ or, PRDM16; or by treatment with BMP7, retinoic acid, FSK, any one or more of the 8 growth factors/inhibitors in the B8 medium (e.g., IGF1, FGF2, BMP7, Rock inhibitor, IBMX, LT3, Dexamethasone, Rosiglitazone), or and combination thereof), reduce, prevent, or discourage a WAT or other alternative non-thermogenic adipocyte phenotype, or a combination thereof. Such programming may include transformation or transfection with nucleic acid constructs that express mRNA/protein, miRNA, siRNA, etc. to genetically program the cells. The nucleic acid constructs can be deployed via, for example, plasmids, viral vectors, CRISPR/Cas9, transient transfection with RNA, etc.

2. Secreted Factor(s)

In some embodiments, secreted factor(s) are administered to a subject in need thereof in an effective amount to treat a disease or condition. In some embodiments, the factor(s) is administered as part of a heterogeneous mixture of factors (e.g., conditioned media, or a fraction isolated therefrom). In some embodiments, one or more specific factors are isolated from conditioned media. In some embodiments, an exosomal factor is administered to a subject in need thereof. The factor(s) can be administered locally or systemic, by for example, parenteral (intramuscular, intraperitoneal, intravenous (IV) or subcutaneous injection) or transdermal injection. The factor(s) are suspended in a carrier for injection. The factor(s) can be formulated in a delivery vehicle or depot.

3. Active Agents

In some embodiments, an active agent is administered to a subject in need thereof in an effective amount to increase the number or activity of thermogenic adipocytes, including endogenous thermogenic adipocytes, in the subject. In some embodiments, the active agent is administered in an effective amount to treat a disease or condition. The active agent compounds can be administered by any suitable means, including those described elsewhere herein, and by other routes and formulations including, but not limited to, pulmonary, nasal, oral (sublingual, buccal), vaginal, and to the rectal mucosa.

Formulations for administration to the mucosa can be incorporated into a tablet, gel, capsule, suspension or emulsion. Standard pharmaceutical excipients are available from any formulator.

Oral formulations may be in the form of chewing gum, gel strips, tablets, capsules, or lozenges. Oral formulations may include excipients or other modifications to the particle which can confer enteric protection or enhanced delivery through the GI tract, including the intestinal epithelia and mucosa (see Samstein, et al., Biomaterials, 29(6):703-8 (2008).

Transdermal formulations may also be prepared. These will typically be ointments, lotions, sprays, or patches, all of which can be prepared using standard technology. Transdermal formulations can include penetration enhancers.

The Examples below describe the results of screening of 3889 compounds for modulation of beige adipocytes. 111 compounds significantly stimulated glycerol release and were subject to further evaluation. Activators of thermogenic adipocytes including FSK and (3-adrenergic agonists such as isoproterenol were identified as primary hits in this screen, confirming the usefulness of the assay. Inhibitors of p38 MAPK and adrenoreceptors that suppress lipolysis were also identified.

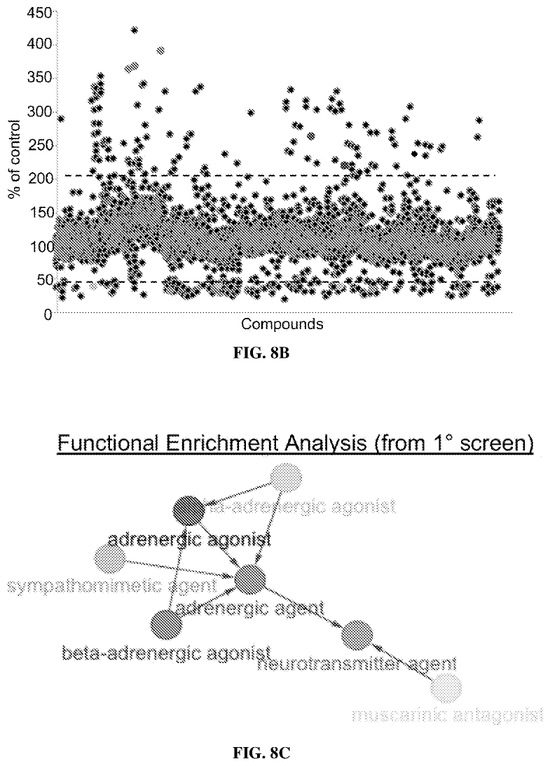

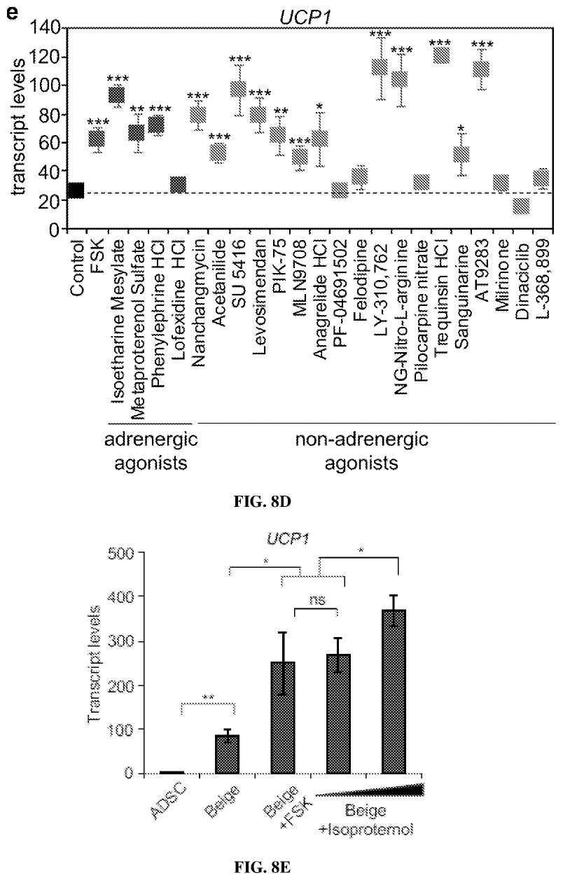

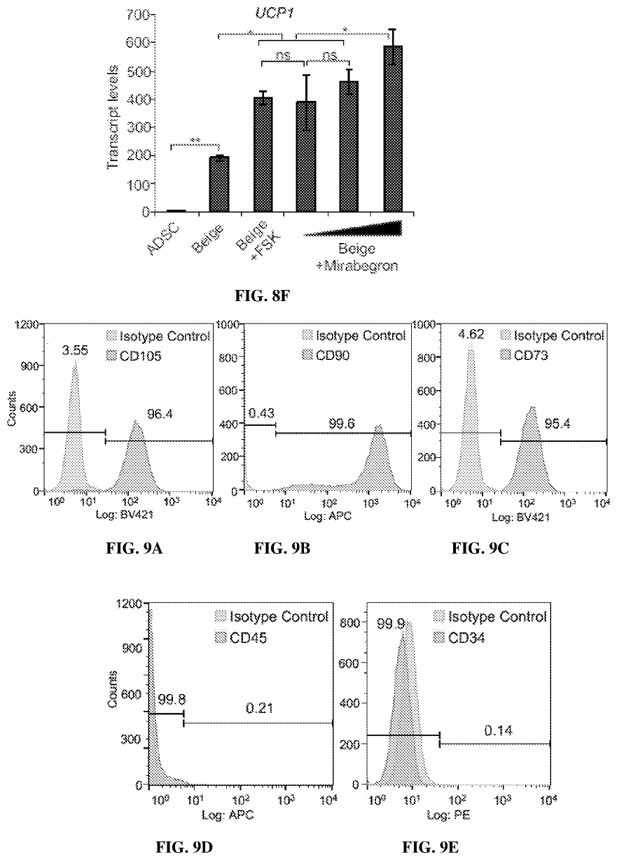

The chemical enrichment analysis tool, BiNChE45 was then used to identify mechanistic links between the 111 preliminary hits identified in this screen, which included one or more alpha-adrenergic agonists, adrenergic angonists, smpathomimetic agents, adrenergic agents, beta-adrenergic agonists, neurotransmitter agents, and muscarinic antagonists (see, e.g., FIGS. 8C and 8D). Thirty-six of the primary screen hits are known adrenergic agonists, indicating that the screening platform is reliably detecting authentic activators of beige adipocytes.

Twenty-four compounds from the primary screen that were also confirmed to increase lipolysis were selected for further analysis. See, e.g., FIGS. 8A-8D and Table 9, which include Forskolin, Isoproterenol HCl, Mirabegron, Isoetharine Mesylate, Metaproterenol Sulfate, Phenylephrine HCl, Lofexidine HCl, Nanchangmycin, Acetanilide, SU 5416, Levosimendan, PIK-75, MLN9708, Anagrelide HCl, PF-04691502, Felodipine, LY-310,762, NG-Nitro-L-arginine, Pilocarpine nitrate, Trequinsin HCl, Sanguinarine, AT9283, Milrinone, Dinaciclib, and L-368,899. Thus, in some embodiments, the subject is treated with an effective amount of one or more of Forskolin, Isoproterenol HCl, Mirabegron, Isoetharine Mesylate, Metaproterenol Sulfate, Phenylephrine HCl, Lofexidine HCl, Nanchangmycin, Acetanilide, SU 5416, Levosimendan, PIK-75, MLN9708, Anagrelide HCl, PF-04691502, Felodipine, LY-310,762, NG-Nitro-L-arginine, Pilocarpine nitrate, Trequinsin HCl, Sanguinarine, AT9283, Milrinone, Dinaciclib, or L-368,899.

In some embodiments, the active agent(s) is an agonist linked to β1-AR, β2-AR, β3-AR, or α3-AR signaling.

To determine if hits identified in the first-round screen activated the thermogenic program, UCP1 transcript levels were used as a readout. This secondary screen incorporated adrenergic agonists, such as Mirabegron, isoproterenol and phenylephrine, and seventeen additional compounds with no reported function in thermogenic regulation (FIG. 8D and Table 9). 17/24 (71%) of the lipolysis-activating compounds identified in the primary screen also activate the thermogenic program through UCP1. Eleven of these compounds have no reported adrenergic agonist activity and may operate through different signaling mechanisms (FIG. 8D and Table 9).

Two phosphodiesterase III (PDE III) inhibitors were identified as activators in both rounds of screening. Other classes of compounds include receptor/channel antagonists (L-368,899, pilocarpine nitrate, LY310-762, Felodipine) and protein kinase inhibitors (Dinaciclib, AT9283, PF-04691502, PIK-75, SU 5416).

Thus, in some embodiments the active agent is a phosphodiesterase III (PDE III) inhibitor or a receptor/channel antagonist such as those provided in FIG. 8D and Table 9.

In some embodiments, the active agent(s) is Trequinisn HCL, Anagrelide, Milrinone, L-368,899, pilocarpine nitrate, LY310-762, Felodipine, Dinaciclib, AT9283, PF-04691502, PIK-75, SU 5416, or a pharmaceutically acceptable salt thereof, or a combination thereof.

B. Conditions to be Treated

The disclosed compositions and methods are particularly useful for treating a subject having a disease, disorder, condition or symptom or comorbidity thereof associated with obesity or excessive weight gain, metabolic disorders such as insulin resistance or diabetes, vascular disease, heart disease, atherosclerosis, dyslipidemia, liver steatosis, loss of physical activity, and loss of endurance. In some embodiments, the compositions and methods can reduce aging or pre-mature aging, increase longevity, increase lifespan, or combination thereof in a subject.

In some embodiments the compositions increase the number or activity or a combination thereof of thermogenic adipocytes in the subject.

1. Metabolic Disorders

The disclosed compositions and methods are useful for treating one or more symptoms or comorbidities of a metabolic disorder, including, but not limited to, insulin resistance, Type 1 or 2 diabetes mellitus, insulin insensitivity, impaired fasting glycaemia, impaired glucose tolerance (IGT), dysglycemia, dyslipidemia, hypertriglyceridemia, hyperglyceridemia, dyslipoproteinemia, hyperlipidemia, hypercholesterolemia, hypolipoproteinemia, and metabolic syndrome.

a. Insulin Resistance and Diabetes

In some embodiments the disclosed compositions are used to treat or prevent insulin resistance or diabetes. Insulin resistance and diabetes can be diagnosed using an oral glucose tolerance test (OGTT). Typically, a fasting patient takes a 75 gram oral dose of glucose. Blood glucose levels are then measured over the following 2 hours. After 2 hours a glycemia less than 7.8 mmol/L (140 mg/dl) is considered normal, a glycemia of between 7.8 to 11.0 mmol/dl (140 to 197 mg/dl) is considered as Impaired Glucose Tolerance (IGT) and a glycemia of greater than or equal to 11.1 mmol/dl (200 mg/dl) is considered diabetes mellitus. An OGTT can be normal or mildly abnormal in simple insulin resistance. A fasting serum insulin level of greater than approximately 60 pmol/L is also considered evidence of insulin resistance.

In some embodiments, the disclosed compositions reduce or decrease fasting blood glucose level, insulin level, or combinations thereof, or reduce, decrease, or delay a rise in fasting blood glucose level, insulin level, or combinations thereof over time. In some embodiments, the compositions disclosed herein delay a rise in fasting blood glucose level, insulin level, or combinations thereof that can occur over time in subjects with high fat diets, little or no exercise, hereditary mutations, hormone changes, advanced age (i.e., becoming elderly), increasing weight or other factors that put them at risk for insulin resistance or diabetes.

b. Metabolic Syndrome

In some embodiments the metabolic disorder is a metabolic syndrome, which typically includes a finding of at least two, preferably three or more of the following symptoms: blood pressure equal to or higher than 130/85 mmHg; fasting blood sugar (glucose) equal to or higher than 100 mg/dL; large waist circumference (length around the waist): Men—40 inches or more, Women—35 inches or more; low HDL cholesterol: Men—under 40 mg/dL, Women—under 50 mg/dL, Triglycerides equal to or higher than 150 mg/dL.

In some embodiments, a method for treating or inhibiting the progression of a metabolic disorder or disease in a subject in need thereof includes administering to the subject an effective amount of a disclosed composition. The subject can display one or more symptoms selected from the group consisting of excessive appetite relative to healthy subjects, elevated blood glucose levels relative to healthy subjects, increased glucose sensitivity relative to healthy subjects, increased glycosylated protein levels relative to healthy subjects, elevated insulin levels relative to healthy subjects, decreased insulin sensitivity relative to healthy subjects, increased blood triglyceride levels relative to healthy subjects, increased blood cholesterol levels relative to healthy subjects, increased blood free fatty acid levels relative to healthy subjects, or a combination thereof. The metabolic disorder or disease can be selected from the list consisting of prediabetes, impaired fasting glycaemia, impaired glucose tolerance (IGT), dysglycemia, insulin resistance, hypertriglyceridemia, hyperglyceridemia, stroke, arteriosclerotic vascular disease (ASVD), dyslipoproteinemia, hypolipoproteinemia, and hyperlipidemia or hypercholesterolemia.

Comorbidities of metabolic disorders include heart disease, vascular disease, atherosclerosis, diabetes, heart attack, kidney disease, nonalcoholic fatty liver disease, peripheral artery disease, and stroke.

In some embodiments, the disclosed compositions are used to prevent, improve, reduce, delay, or improve one or more symptoms or comorbidities of metabolic disorder.

Methods of treating a metabolic disorder can also include dietary modifications such as reduced fat, increased fruits, vegetables, and whole-grain products, increase fish or fish oils; increased exercise; weight loss; managing blood pressure and blood sugar; and not smoking. Combination therapies can include administration of the compositions disclosed herein in combination with a second therapeutic agent that is known in the art for treating insulin resistance, Type 1 or 2 diabetes mellitus, high cholesterol, high blood lipids, metabolic syndrome, or a symptom of comorbidity thereof. For example, the compositions can be administered in combination with insulin or a cholesterol-lowering drug. Cholesterol lowering drugs include, but are not limited to, statins such as atorvastatin (Lipitor), simvastatin (Zocor), lovastatin (Mevacor), pravastatin (Pravachol), and rosuvastatin (Crestor).

2. Weight Gain and Obesity

In some embodiments, the disclosed compositions are used to reduce or decrease, total body weight in a subject. The disclosed compositions can also be used to reduce, decrease, or delay a rise in total body weight over time. In some embodiments, the compositions disclosed herein delay a rise in total body weight, for example, that which can occur over time in subjects with high fat diets, little or no exercise, hereditary mutations, hormone changes, advancing age (i.e., elderly), diabetes, high cholesterol or high triglycerides.

The disclosed compositions and methods can be used for treating or preventing obesity or one or more symptoms or comorbidities thereof.

In some embodiments the subject is a healthy individual of normal weight, or is already overweight, and any additional weight gain could result in obesity or obesity-associate comorbidities. Body Mass Index is a standardized method of determining a subject's weight category using a calculus that is known in the art. A subject can be, for example, underweight: BMI of less 18.5; normal weight: BMI of 18.5-24.9; overweight: BMI of 25-29.9; or obese: BMI of 30 or greater. Therefore, in preferred embodiments, the disclosed compositions are useful for treating or preventing weight gain in a subject with a normal BMI, an overweight BMI, or an obese BMI. For example, the disclosed compositions can be used to treat or prevent weight gain in a subject with a BMI of about 25, 26, 27, 28, 29, 30, or more.

3. Endurance and Physical Activity

The disclosed compositions and methods are useful for increasing or improving physical activity; increasing or improving endurance; or combinations thereof compared to, for example, a matched, untreated subject. The terms “endurance” or “stamina” as used herein mean the ability or strength to continue or last, especially despite fatigue, stress, or other adverse conditions. Therefore, in some embodiments, a subject treated with the disclosed compositions continues or persists in a physical activity longer compared to a control. In some embodiments, a subject treated with disclosed compositions completes a physical activity faster compared to a control. Controls can include, for example, the subject prior to treatment, or an untreated subject. Physical activities include, but are not limited to, walking, jogging, running, biking, swimming, and lifting. Increased endurance can include an increase in the duration of the physical activity, or an increase in intensity of the physical activity over the same duration. In some embodiments, the disclosed compositions are used to reduce or delay a decline in endurance or physical activity over time, for example with increasing age or in an elderly subject.

In some embodiments, the disclosed compositions are used to reduce or decrease, fatigue in a subject. In some embodiments, the disclosed compositions are used to reduce, decrease, or delay fatigue in a subject over time. As used herein “fatigue” can also include weakness, exhaustion, lethargy, languidness, languor, lassitude and listlessness.

4. Hypertension

The disclosed compositions and methods can be used to treat or prevent the development or progression of high blood pressure or hypertension. Primary hypertension has no known cause. The methods and compositions disclosed herein can be combined with other methods of treating and preventing of hypertension including maintaining normal body weight, reducing dietary sodium intake, engaging in regular aerobic physical activity such as brisk walking, limiting alcohol consumption, consuming a diet rich in fruit and vegetables, consuming a diet with reduced content of saturated and total fat, and combinations thereof.

The disclosed compositions may aid, augment, replace or supplement such lifestyle changes such as increased physical activity, maintain or achieve normal body weight, and lower blood lipids in a similar way a changed diet may.

Insulin resistance and obesity are known risk factors of hypertension. Disclosed compositions may reduce weight increases and insulin resistance and may hence reduce the risk of developing hypertension, or may reduce blood pressure in a patient with hypertension, insulin resistance, obesity, or combinations thereof.

C. Combination Therapy

Any of the disclosed compositions can be used in any combination. In some embodiments, the methods include administration of one or more of the disclosed compositions in combination with one or more additional active agents. The additional active agent can be, for example, a traditional therapy for the disease or disorder being treated or another compound that induced or increases thermogenic adipocytes.

The term “combination” or “combined” is used to refer to either concomitant, simultaneous, or sequential administration of two or more active ingredients. The combinations can be administered either concomitantly (e.g., as an admixture), separately but simultaneously (e.g., via separate intravenous lines into the same subject; one agent is given orally while the other agent is given by infusion or injection, etc.), or sequentially (e.g., one agent is given first followed by the second).

D. Methods of Screening

Non-naturally occurring thermogenic adipocytes including GC are a useful cell source for performing screening applications.

For example, the culture conditions and compositions for non-naturally occurring thermogenic adipocytes including GC can be fully-defined and serum-free system. Thus, factors/inhibitors may be removed or added to modulate the differentiation process in a high-throughput screening approach to look for compounds that promote or activate beiging and thermogenesis.

In some embodiments, screening assays can include random screening of large libraries of test compounds. Alternatively, the assays may be used to focus on particular classes of compounds suspected of modulating the function or expression of adipocytes. For example, the screening methods can be used to identify drugs that modulate differentiation, activate beige adipocytes (e.g., drugs that have an effect similar to FSK), etc.

Assays can include determinations of protein expression, protein activity, or binding activity of one or more marker of adipocytes, including the markers of BAT, beige cells, non-naturally occurring thermogenic adipocytes including GC, and WAT disclosed herein or otherwise known in the art. Other assays can include determinations of nucleic acid transcription or translation, for example mRNA levels, miRNA levels, mRNA stability, mRNA degradation, transcription rates, and translation rates of the markers including those discussed herein. For example, in some embodiments, mRNA or protein levels of ICAM1, MMP3, UCP1, DIO2, or a combination thereof is modulated. Assays can also include examining cell structure and histology, respiration levels (e.g., basal respiration, maximal respiration, proton leak, etc.), and other assays discussed herein.

Test assays can include standard methods such as FACS, FACE, ELISA, Northern blotting, Western blotting, qRT-PCR, RNA-Seq, immunostaining, etc.

Other test assays include, for example, lipolysis assay, glycerol release, identification/comparison of secreted factors, use of thermogenic or other dyes such as mitochondrial dyes or membrane potential dyes, and others disclosed herein and elsewhere for screening for changes or alterations in adipocyte differentiation, activation, etc.

Techniques for high throughput screening of compounds are known in the art. Large numbers of small peptide test compounds can be synthesized on a solid substrate, such as plastic pins or some other surface. Round polypeptide can be detected by various methods. As illustrated in the Examples below, libraries of small molecule compounds are also available.

Non-naturally occurring thermogenic adipocytes (including GC) differentiation systems can also be used with patient samples for autologous transplantation or to model diseases. For example, the presence of thermogenic adipocytes are known to be considerably reduced in obese individuals. A differentiation system could therefore be used to evaluate differences in functional activity between thermogenic adipocytes established from lean or obese individuals, in order to identify genetic variants and polymorphisms related to obesity or obesity-associated diseases.

### EXAMPLES

### Example 1: Differentiation and Molecular Characterization of GlucoCytes

Materials and Methods

Cell Culture and Differentiations

ADSCs (ThermoFisher, cat no: R7788115, Lot #: 1001001 and Lot #1001002; ATCC, ASC52telo, cat no: ATCC SCRC-4000) were grown in ADSC-growth medium comprising 10% fetal bovine serum (Atlanta Biologicals, S10250) in DMEM (Corning, 10013CV) with 1× Antibiotic-Antimycotic (Corning, 30-004-CI), 1×MEM non-essential amino acids (Corning, 25-025-CI), 1× Glutagro (Corning, 25-015-CI) and 1×BME (ThermoFisher, 21985023). Cells were seeded at a density of 5000 cell/cm² and passaged at 80-90% confluency, approximately 5 days post-plating. To passage cells from a 100 mm cell culture plate, ADSCs were first washed with DPBS (Corning, 21031CV) and then incubated with 5 ml of Accutase (Innovative Cell Technologies, AT104) for 5 minutes at room temperature. Next, 5 ml of DPBS was added and cells were centrifuged at 1000 rpm (200×g) for 4 min in a swinging bucket centrifuge. Cells were resuspended in ADSC-growth medium and counted using a hemocytometer for seeding. Following cell seeding, media was changed every other day. Additional ADSC lines purchased from Lonza, PT-5006 (Lot 0000543947 and 18TL215666); PT-5008 (1F4521 and 1F4619) were grown using the ADSC Bulletkit and ReagentPack (PT-4505 and CC-5034) according to manufacturer's instructions.

GC differentiation of ADSCs were performed using B-8 medium, which included the addition of a cocktail of 8 growth factors or inhibitors added to a chemically-defined based medium (DM). DM was composed of DMEM/F-12 w/o glutamine supplemented with 2% Probumin (EMD Milipore, 821005), 1× Antibiotic-Antimyotic (Corning, 30-004-CI), 1×MEM non-essential amino acids (Corning, 25-025-CI), 1× Trace Elements A (Corning, 99-182-CI), 1× Trace Elements B (Corning, 99-175-CI), 1× Trace Elements C (Corning, 99-176-CI) 50 μg/mL ascorbic acid (Sigma, A8960), 10 μg/mL Transferrin (Athens Research and Technology, 16-16-A32001-LEL), 0.1 mM 2-mercaptoethanol (Gibco, 21985023) and 1× Glutagro (Corning, 25-015-CI). B-8 medium was generated by adding the following 8 items to DM, 200 ng/mL LONG® R3 human IGF-I (Sigma, 85580C), 8 ng/mL human basic-FGF (R&D Systems, 4114-TC), 100 ng/mL human-BMP7 (R&D Systems, 354-BP/CF), 10 μM Y27632 (R&D Systems, 1254/50), 2 μM Rosiglitazone (R&D Systems, 5325/50), 1 nM Triiodo-L-thyronine (Sigma, 64245-250MG), 1 μM Dexamethasone (R&D Systems, 1126/100) and 500 μM Isobutylmethylxanthine (Sigma, I5879-5G). To perform GC differentiations (i.e., generate beige adipocytes), ADSCs were passaged and seeded at a density of 5000 cell/cm² and were grown to confluency, ˜5 days. B-8 medium was subsequently added and medium was changed every other day for 3 weeks.

TABLE 4
Composition of B-8 Media.

| | | | Stock | | | | | | Stock | |
|---|---|---|---|---|---|---|---|---|---|---|
| Compound | Vender | Cat# | Concen. | | Diluent | Final Conc. | | Medium | Storage | |
| Probumin-BSA | EMD | 821005 | 20% | | DMEM/F-12 | 2% | | Base | −20 | C. |
| | Milipore | | | | | | | medium | | |
| Pen/Strep/anti- | FisherSci | MT30004CI | 100X | | — | 1X | | Base | −20 | C. |
| mycotic | | | | | | | | medium | | |
| Nonessential | Fisher Sci | MT25025CI | 100X | | — | 1X | | Base | 4 | C. |
| amino acids | | | | | | | | medium | | |
| GlutaGro | Fisher Sci | MT25015CI | 100X | | — | 1X | | Base | 4 | C. |
| | | | | | | | | medium | | |
| Trace Elements A | FisherSci | MT99182CI | 1000X | | — | 1X | | Base | 4 | C. |
| | | | | | | | | medium | | |
| Trace Elements B | FisherSci | MT99175CI | 1000X | | — | 1X | | Base | 4 | C. |
| | | | | | | | | medium | | |
| Trace Elements C | FisherSci | MT99176CI | 1000X | | — | 1X | | Base | 4 | C. |
| | | | | | | | | medium | | |
| L-ascorbic acid | Sigma | A8960-5G | 50 | mg/ml | ddH2O | 50 | ug/ml | Base | 4 | C. |
| | | | | | | | | medium | | |
| Transferrin | Athens | 16-16- | 10 | mg/ml | DMEM/F-12 | 10 | ug/ml | Base | 4 | C. |
| | Research | 032001-LEL | | | | | | medium | | |
| | Technology | | | | | | | | | |
| DMEM/F-12 | Fisher Sci | MT15090CM | 1X | | — | 1X | | Base | 4 | C. |
| | | | | | | | | medium | | |
| BMP7 | R&D | 354-BP/CF | 100 | ug/ml | 4 mM HCL + | 100 | ng/ml | B-8 | −20 | C. |
| | Systems | | | | 0.2% Probumin | | | | | |
| IGF-1 | Sigma | 85580C- | 1 | mg/ml | 4 mM HCl + | 200 | ng/ml | B-8 | −20 | C. |
| | (SAFC) | 50MG | | | 0.2% BSA | | | | | |
| FGF2 | R&D | 4114-TC | 25 | ug/ml | PBS + | 8 | ng/ml | B-8 | −20 | C. |
| | Systems | | | | 0.2% BSA | | | | | |
| Y-27632 | R&D | 1254/50 | 10 | mM | DMSO | 10 | uM | B-8 | −20 | C. |
| | Systems | | | | | | | | | |
| Rosiglitazone | R&D | 5325/50 | 20 | mM | DMSG | 2 | uM | B-8 | −20 | C. |
| | Systems | | | | | | | | | |
| Dexamethasone | R&D | 1126/100 | 10 | mM | DMSO | 1 | uM | B-8 | −20 | C. |
| | Systems | | | | | | | | | |
| Triiodo-L- | Sigma | 64245- | 20 | uM | DMSO | 1 | nM | B-8 | −20 | C. |
| thyronine (LT3) | | 250MG-M | | | | | | | | |
| IBMX | Sigma | I5879-5G | 500 | mM | DMSO | 500 | uM | B-8 | −20 | C. |

To perform white adipocyte differentiations, ADSCs were passaged and seeded at a density of 5000 cell/cm², grown to confluency, and differentiated using the StemPro adipogenesis differentiation kit (ThermoFisher, A10070-01). Media was changed every two days for 3 weeks.

Immunostaining

Before fixation, live cells were treated with MitoTracker™ Deep Red FM (ThermoFisher, M22426) for 45 mins in cell culture incubator. Next, cells were washed in DPBS three times and fixed in 4% formaldehyde (ThermoScientific, 28906) in a DPBS solution for 15 minutes. Cells were blocked in DPBS based block solution containing 10% donkey serum (Equitech-Bio, SD30-0500), 0.2% Saponin (EMD Millipore, 558255-100GM) and 0.3 M Glycine (Sigma, G7126-1KG) for 1 hour at room temperature. Primary antibodies UCP-1 (Abcam, Ab10983) or Perilipin-1 (Cell Signaling Technology, D1D8) where prepared in DPBS based primary antibody incubation solution containing 10% Donkey serum and 0.2% Saponin at dilutions of 1:500 and 1:200, respectively, and incubated overnight at 4° C. After 3×5-minute washes in 0.2% Saponin-DPBS solution, cells were incubated with secondary antibodies, Alexa Fluor 555 donkey anti-Rabbit IgG (H+L) (ThermoFisher, A31572) in DPBS based secondary antibody incubation solution containing 2.5% Donkey Serum and 0.2% Saponin at dilutions of 1:250 for 1 hour at room temperature in the dark. Following secondary antibody incubation, cells were washed twice with DPBS for 5 minutes each and then added HCS LipidTOX™ Green Neutral Lipid Stain (ThermoFisher, H34475) at 1:200 dilution in DPBS for 30 minutes. Subsequent to neutral lipid staining, cells were treated with DAPI (Sigma, D9542) for 5 minutes. Following additional 3 washes in DPBS, cells were treated with ProLong Gold mounting medium (ThermoFisher, P36934). Samples were cured for 24 hours at room temperature in the dark and later used for imaging on an Olympus FV1200 confocal microscope.

Quantitation of beige adipocytes was performed using manual counting with the Cell Counting tool on ImageJ, following immunostaining with UCP1, DAPI and LipidTox Green of beige adipocytes. Six independent fields of view were assessed in a three-step quantitation process. First, every DAPI signal was counted to determine the total number of cells in each field. Second, the UCP1 and LipidTox Green and DAPI channels were overlaid. Importantly, a conservative approach was applied here as dim staining was considered ‘negative’ and is likely to underestimate the % of UCP1+ cells. Third, the total number of UCP1+LipidTox Green+ double positive cells (DAPI+) were used to calculate the total percentages.

Flow cytometry was performed on ADSCs, by incubating 0.5 million cells for 30 minutes at 4° C. with the following antibodies: CD73-BV421 (BD Biosciences, 562431), CD105-BV421 (BD Biosciences, 563920), IgG1 Isotype-BV421 (BD Biosciences, 562438), CD45-PE (BD Biosciences, 555483), IgG1 Isotype-PE (BD Biosciences, 555749), CD34-PE (R&D Systems, FAB7227P), CD90-APC (eBiosciences, 17-0909-42). Cells were washed in PBS and analyzed on a Beckman Coulter CyAn.

Immunoblotting

To prepare whole cell lysates for immunoblotting, approximately 20 million cells were washed with cold DPBS and harvested by scraping with 300 μl cold RIPA buffer (Sigma, R0278) containing 2% SDS (KD Medical, RGE-3235), 1× Complete, Mini, EDTA-free protease inhibitor cocktail (Roche, 11836170001) and 1× phosphatase inhibitor cocktail set II (EMD Millipore, 524625). Cell lysates were heated for 10 minutes at 95° C. and centrifuged at 13,000×g for 10 minutes at 4° C. Supernatants were removed carefully to avoid any lipid precipitates and transferred to a new tube. Protein concentration was determined using the RC DC protein assay kit I (Bio-Rad, 5000121). Samples (25 μg total protein) were mixed with 2× Laemmli sample buffer (Bio-Rad, 1610737) and loaded onto a Bolt 4-12% Bis-Tris polyacrylamide gel (ThermoFisher, NW04122BOX). Samples were electrophoresed for 40 minutes using Bolt MES SDS Running buffer (ThermoFisher, B0002) and blotted onto a 0.45 μm PVDF membrane (Bio-Rad, 1620260) for 1 hour in Bolt transfer buffer (ThermoFisher, BT00061). Membranes were blocked in 5% non-fat dry milk (Bio-Rad, 1706404) in 1× Tris-buffered saline (FisherSci, BP2471) with 0.05% Tween-20 (Sigma, P7949) (TBST). Membranes were incubated with primary antibody at 1/1000 dilution, overnight at 4° C. in blocking solution. Primary antibodies were UCP1 (Abcam, Ab10983), p-HSLS660 (Cell Signaling, #4126), HSL (Cell Signaling, #4107), p-CREBS133 (Cell Signaling, #9191), p-P38MAPKT180/Y182 (Cell Signaling, #9216), P38MAPK (Cell Signaling, #9212), and Cofillin (Santa Cruz, sc-376476 HRP). The following day, blots were washed 3 times in TBST for 5 minutes each, incubated with secondary antibody, anti-rabbit-HRP or anti-mouse HRP (Dako, P0448; Dako, P0260) for 1 hour in blocking solution and subsequently washed for 5 minutes in TBST prior to developing. Amersham ECL western blotting detection reagent (GE Healthcare, RPN2106) was used for detection purposes.

RNA Transcript Analysis (qRT-PCR and RNA-Seq Analysis)

Cells were washed with DPBS, lysed directly on the plate and used for RNA isolation with the E.Z.N.A Total RNA Kit (Omega, R6834-02) according to manufacturer instructions. RNA quantitation was performed with the Biotek Synergy 2 plate reader and cDNA was synthesized using 1 ug of RNA with the iScript cDNA Synthesis Kit (Bio-Rad, 1708841). The qRT-PCR was performed on a ViiA7 Real-Time PCR System (ThermoFisher) using a TaqMan Universal PCR Master Mix (ThermoFisher, 4324020) according to manufacturer instructions with the following TaqMan primer/probe sets (UCP1, Hs01084772_m1), DIO2, Hs00988260_ml, 18S rRNA, Hs03928985_g1). Each assay was performed in triplicate and normalized to 18S rRNA and graphed as mean+/−standard deviation.

For samples prepared for RNA-Seq, total RNA samples were isolated using E.Z.N.A Total RNA kit and were DNase treated to remove potential genomic DNA contamination (Omega, E1091). RNA sample with an RIN>9 were processed for RNA-Seq. An average of 30 million paired-end reads with a length of 75 bp were generated per library on a NextSeq platform by Georgia Genomic Facility. Reads were mapped to the human genome (hg19) by STAR v2.5.3a using default setting and read counts were obtained in STAR quant-mode (Dobin et al., Bioinformatics, 1; 29(1):15-21 (2013). doi: 10.1093/bioinformatics/bts635). Gene expression analysis was preformed using limma, Glimma and edgeR in R Studio (Law, et al., F1000Research 2016, 5:1408 Last updated: 20 Aug. 2018). Log-2 normalized counts per million read were used for generating heatmap. The ADSC-derived beige cells were compared to downloadeddatasets (GEO: GSE78647, GSE78544, GSE78568, GSE78535, GSE78528, GSE78623, GSE78608, GSE78607, GSE57896, GSE59703; and EBI: E-MTAB2602). Principal component analysis of adipose derived stem cells used in this study and other relevant cell types was performed in R package, DESeq2 with its plot PCA function (Love, et al., Genome Biol 15, 550 (2014)). R package ‘ggplot2’ was used to better visualize the result (Wickham, H. in Use R!, Edn. Second edition. 1 online resource (Springer, Switzerland; 2016). The ADSCs were compared to downloaded datasets (GEO: GSM909310, GSE78615, GSE90271, GSE78609, GSE78635, GSE80095, PRJNA449980, PRJNA277616, PRJNA277616, GSE59703, GSE113764, GSE101655).

Electron Microscopy

ADSCs were differentiated to GC on 4-well chamber slides (Nunc) for 21 days. Samples were fixed in 2.5% glutaraldehyde (v/v) and 5 mM CaCl2 in 0.1 cacodylate buffer, washed in DPBS and post-fixed for 1 hour in 1% OsO4 in cacodylate buffer with 5 mM CaCl2 and 0.8% potassium ferricyanide. Samples were dehydrated in acetone, Epon embedded and sectioned. Sections were stained with uranyl acetate and lead citrate and TEM analysis was performed on a JEOL 1210 electron microscope. For SEM, analysis was performed on an FEI Teneo scanning emission electron microscope.

Quantification and Statistical Analysis

One-way ANOVA was used to evaluate statistical significance for Seahorse assays, qRT-PCR and lipolysis assays. Unless otherwise indicated, an n=3 for each independent replicate was used Mann-Whitney rank sum tests were used to evaluate the significance of the indirect calorimetry. A two-tailed Student's t-test was used for all other assays. A p-value of <0.05 was considered statistically significant.

Results

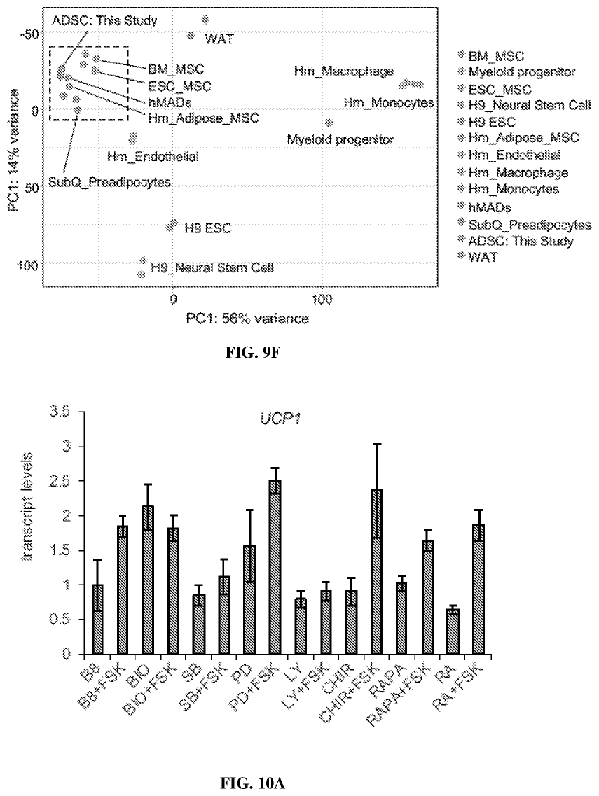

ADSCs used in this study were characterized and validated by analysis of cell surface markers (FIG. 9A-9E) and RNA-seq analysis (FIG. 9F). This analysis confirmed that cells used in this study are comparable to equivalent cells reported in the literature.

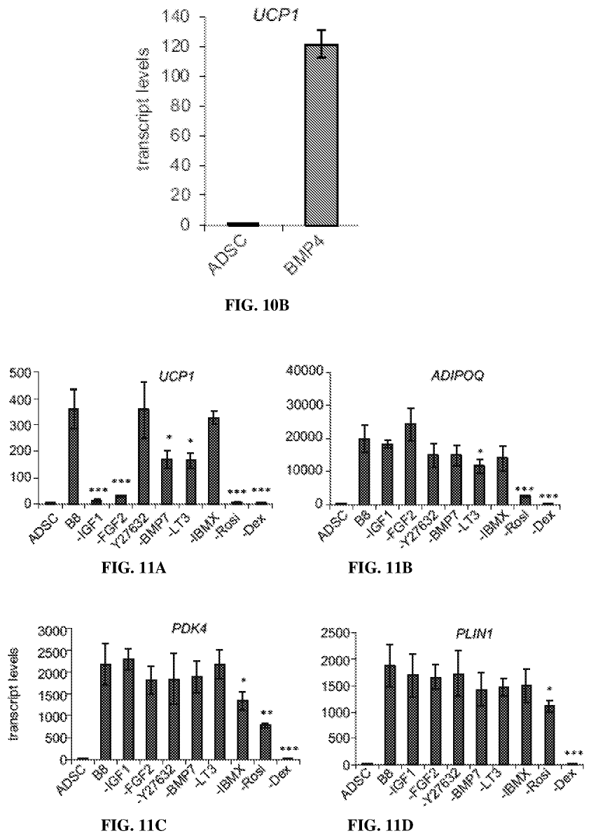

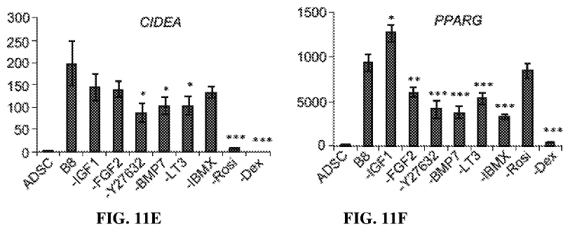

Previous studies have demonstrated that thermogenic adipocytes can be generated from ADSCs, but their efficiency, molecular characterization, and in vivo functionality have not been characterized in detail. To obtain a highly-enriched beige adipocyte population from human ADSCs, a chemically-defined media was developed by testing factors individually or combinatorially for pro-thermogenic activity in basal medium (Menendez, et al., Proc Natl Acad Sci USA 108, 19240-19245 (2011), Singh, et al., Cell Stem Cell 10, 312-326 (2012), Wang, et al., Blood 110, 4111-4119 (2007), Cliff, et al., Cell Stem Cell 21, 502-516 e509 (2017)) (FIGS. 10A, 10B, 11A-11F), Table 5).

TABLE 5
Summary of effect of growth factors and small molecules
on beige adipocyte differentiation and cell survival

| | Enhanced beige | |
|---|---|---|
| Factor/small | differentiation | Improved cell |
| molecule | (UCP1 transcript) | viability/survival |
| IGF1 (200 ng/ml) | +++ | +++ |
| FGF2 (8 ng/ml) | +++ | + |
| Y27632 (10 μM) | − | + |
| BMP7 (100 ng/ml) | ++ | − |
| BMP4 (50 ng/ml) | ++ | − |
| All-trans retinoic | − | − |
| acid (4 μM) | | |
| PD0325901 (1 μM) | − | − |
| BIO (2 μm) | − | − |
| CHIR99021 (10 μM) | − | − |
| LY294002 (50 μM) | − | − |
| Rapamycin (250 nM) | − | − |
| SB431542 (20 μM) | − | − |

This media, including 8 important growth factors and inhibitors, termed Beiging-8 (B-8) medium, results in high-efficiency differentiation to a cell type referred to herein as GlucoCytes (GC) and beige adipocytes (e.g., ADSC-derived beige adipocytes). Like their in vivo counterparts in BAT, GC have multi-locular lipid droplets surrounded by an abundance of mitochondria.

TABLE 6
Media compositions from previous studies from ADSC-derived beige adipocytes

| | | Wang, Biochem |
|---|---|---|
| | Bartesaghi, Mol | Biophys Res |
| This Study (B-8) | Endocrinol 29, 130-139 (2015) | Comm. 478, (2016) |
| DMEM/F-12 | DMEM-F12 | α-MEM |
| Probumin-albumin (2%) | 3% fetal calf serum | 10% fetal bovine serum |
| Nonessential amino acids (1X) | Dexamethasone (100 mM) | Dexametasone (100 mM) |
| GlutaGro (1X) | 3-isobutyl-1-methyxanthine (500 μM) | Ascorbate-2-phosphate (50 μg/ml) |
| Trace Elements A (1X) | insulin (0.85 μM) | insulin (10 μg/ml) |
| Trace Elements B (1X) | Triiodothyronine (5 nM) | Triiodothyronine (1 nM) |
| Trace Elements C (1X) | Rosiglitazone (100 nM) | indomethacin (50 μg/ml) |
| L-ascorbic acid (50 μg/ml) | | forskolin (10 μM) |
| Transferrin (10 μg/ml) | | |
| IGF-1 (200 ng/ml) | | |
| FGF2 (8 ng/ml) | | |
| Rosiglitazone (2 μM) | | |
| Dexamethasone (1 μM) | | |
| Triiodothyronine (1 nM) | | |
| BMP7 (100 ng/ml) | | |
| 3-isobutyl-1-methyxanthine (500 μM) | | |
| Y-27632 (10 μM) | | |
| Differentiation Efficiency | Differentiation Efficiency | Differentiation Efficiency |
| >90% | Not determined/described | Not determined/described |
| NOTE: Defined base media | | NOTE: 3-isobutyl-1-methyxanthine |
| components are italicized and B-8 | | (0.45 mM) was substituted for |
| factors/inhibitors are bolded | | forskolin in some experiments |

To establish if GC have a molecular signature consistent with them being thermogenic adipocytes, transcript levels UCP1 and DIO2 were determined in relation to ADSCs. Both transcripts were elevated in GC and were further induced following by forskolin (FSK), which activates thermogenesis through adenylyl cyclase by increasing intracellular levels of cAMP (FIG. 2A) (Bronnikov et al., J Biol Chem 267, 2006-2013 (1992); Schimmel et al., Am J Physiol 248, E224-229 (1985)). In the resting state, beige adipocytes express 170- and 15-fold higher levels of UCP1 and DIO2 transcripts, respectively, compared to ADSCs. Stimulation with FSK, further increased levels of UCP1 and DIO2 transcripts by 520- and 130-fold compared to ADSCs, respectively. These observations are consistent with the response bona fide thermogenic adipocytes can have to activated cAMP-dependent signaling (Shinoda, et al., Nat Med 21, 389-394 (2015)).

UCP1 expression in GC was then confirmed to overlap with mitochondria and uniformly surrounded multilocular lipid droplets. The fully-defined, serum-free B-8 media used, supported the conversion of ADSCs to UCP1+GC with efficiencies that are typically >90%, (see, e.g., Table 7, FIG. 2B) are LipidTox Green+ after 21 days.

TABLE 7
Quantitation of GC in 6 independent fields of view at 10X
magnification. The total number of cells was determined
by counting DAPI. UCP1 surrounding DAPI indicated a GC.
Percentage positive of each field of view is indicated.

| Field | 1 | 2 | 3 | 4 | 5 | 6 |
|---|---|---|---|---|---|---|
| Total | 805 | 813 | 803 | 791 | 795 | 782 |
| Non-Positives | 53 | 69 | 58 | 65 | 62 | 59 |
| Positives | 752 | 744 | 745 | 726 | 733 | 723 |
| Percentage | 93.4% | 91.5% | 92.8% | 91.7% | 92.2% | 92.4% |

The B-8 method was compared to the previously described methods by Bartesaghi et al., Mol Endocrinol 29, 130-139 (2015) and Wang et al., Biochem Biophys Res Commun 478, 689-695 (2016) (FIG. 2E, 2F). <30% of cells generated by these latter approaches expressed UCP1, indicating that the majority of cells in these cultures are not beige adipocytes. Cells showed clear temporal changes in the expression of general adipocyte markers under B-8 differentiation conditions followed by increased levels of UCP1 mRNA, consistent with them transitioning from a general pre-adipocyte state to a thermogenic, beige adipocyte state (FIG. 2G-2N).

The efficacy of beige cell differentiation with B-8 medium was confirmed using six, independent human ADSC primary cell lines. Efficient differentiation of ADSCs to a beige state occurred independently of passage number, gender of the donor or, body mass index and T2D status of donors (FIGS. 2C, 2D, 2O-2T).

The efficacy of GC differentiation with B-8 medium was confirmed using two additional ADSC lines, isolated from 2 additional donors (FIGS. 2C and 2D). An additional line purchased from ThermoFisher, show high efficiency differentiation to GC and induction of UCP1 following FSK (20 uM) treatment by qRT-PCR (FIG. 2C). An immortalized ADSC line purchased from ATCC (ASC52-telo) resulted in high efficiency differentiation to GC and induction of UCP1 transcript levels by qRT-PCR (FIG. 2D). Transmission electron microscopy and scanning electron microscopy was consistent with GC being similar to brown adipocytes based on their high mitochondrial content and multilocular lipid composition.

Next, ADSCs were differentiated to GC with B-8 medium and molecular analysis performed by RNA-sequencing (RNA-seq). Comparison of global RNA signatures from GC, derived from ADSCs, showed that they shared a common molecular signature with BAT (FIGS. 3A, 3C, 3D, 3G) and a closer similarity to thermogenic adipocytes (BAT or beige cells), than to other cell types, by hierarchical cluster analysis (FIG. 3B, 3F).

Hierarchical clustering analysis of RNA-seq data show that ADSC-derived beige adipocytes cluster closely with other human thermogenic adipocytes, including human brown ((Shinoda, et al., Nat Med 21, 389-394 (2015)) and beige (Loft, et al., Genes Dev 29, 7-22 (2015), Moisan, et al., Nat Cell Biol 17, 57-67 (2015)) adipocytes. These different sources of thermogenic adipocytes segregate away from other human cell types included in this analysis (Encode Project Consortium, Nature 489, 57-74 (2012)) (FIG. 3F).

Furthermore, the cells could be activated by forskolin, similar to brown adipose cells (FIGS. 3C, 3D). Transcripts for thermogenic genes such as UCP1, IRF4, PPARGC1A and PDK4 are shown to be elevated following FSK (20 μM, 6 hours) treatment. Comparing global gene expression signatures in beige and brown adipocytes showed a high correlation under unstimulated and FSK-treated conditions (FIG. 3G, 2E, 2F).

Beige adipocytes exhibit elevated levels of thermogenic markers, compared to that in white adipocytes and ADSCs (FIGS. 3E, 3H). In addition, levels of these thermogenic adipocyte marker were upregulated in beige cells following induction with FSK (FIG. 3H).

The gene expression profiles from RNA-seq data of GC were compared to all known brown and beige adipocytes from in vivo and in vitro sources, and found that GC express unique markers to all other thermogenic cells (FIG. 4). These data indicate that GC are a unique thermogenic cell type that has not been previously developed in vitro, or that exists in vivo.

The ‘browning probability score’ using ProFAT, a computational assessment tool (Cheng, et al., Cell Rep 23, 3112-3125 (2018)), that combines 97 human adipose microarray and RNA-seq datasets from various sample types to identify a common expression signature for white and brown adipocytes. The brown adipocyte signature identified by ProFAT analysis can then be used to derive a brown adipocyte correlation value that is an indicator of brown adipocyte identity. When RNA-seq data from ADSC-derived beige cells was applied to ProFAT, a browning probability coefficient of 0.98 was obtained (FIG. 3I, 3J), indicative that these cells are thermogenic adipocytes. This correlation value exceeds that assigned to human brown adipocytes derived from immortalized pre-adipocytes (Shinoda, et al., Nat Med 21, 389-394 (2015)) (FIG. 3I). The phenotypic and molecular characteristics of these cells are consistent with authentic beige adipocytes.

Taken together, these data indicate the development of a robust and efficient platform for the differentiation of GC from ADSCs in vitro. The GC phenotypically and genetically resemble thermogenic adipocytes in vivo, in regards to the presence of multilocular lipid droplet formation, high mitochondrial content and thermogenic markers, such as UCP1.

The supernatant were also collected from day 20 GC cultures and extracellular vesicles were collected by high-speed ultracentfugation and imaged by TEM. Results show that GC can secrete extracellular vesicles, such as exosomes.

Uniform perilipin expression was also found in beige adipocytes.

Since B-8 medium could generate GC from ADSCS, the possibility that B-8 media could direct the efficient conversion of white adipocytes to GC was investigated (Barquissau et al., Mol Metab 5, 352-365 (2016); Rosenwald et al., Nat Cell Biol 15, 659-667 (2013); Rosenwald and Wolfrum, Adipocyte 3, 4-9 (2014)). ADSCs were differentiated to white adipocytes (WA) using a commercially available media for 14 days and then, treated with B-8 medium for an additional 14 days. Following treatment with B-8 media, transcripts for thermogenic markers, such as UCP1, were significantly upregulated by RNA-seq analysis compared to WA (FIG. 3E), indicating that B-8 media promotes the “browning” of white adipocytes.

### Example 2: GC Exhibit Uncoupled Respiration and Thermogenic Activity

Materials and Methods

Seahorse Assay

To perform the assay, ADSCs were plated on XFe24 plates (Agilent) at 5000 cell/cm² and grown to confluency. Cells were differentiated to GC or WA as described above. The XF Cell Mito Stress Test Kit (Seahorse Bioscience, 103015-100) was used to perform the assay and performed according to manufacturer instructions. Briefly, Oligomycin (2 μM), FCCP (2 μM), and rotenone/antimycin a (5 μM each) were used during the assay at indicated time points, following analysis based on their titration. One day before the assay, cells were stimulated with forskolin (20 μM) for 24 hours in freshly prepared XF assay medium (Seahorse Biosciences, 102353-100) containing 25 mM glucose, 1× Glutagro and 1 mM sodium pyruvate. At 1 hour prior to the assay, cells were washed 3 times in freshly prepared XF assay medium and the medium was given to the cells as they were placed in a non-CO₂ incubator at 37° C. Following the assay, cells were lysed using RIPA buffer and protein concentration was determined by Bradford assay. Data was normalized by protein concentration and analyzed in Wave software (Agilent).

Lipolysis Assay

ADSCs were seeded at 5000 cell/cm² in 12-well plates, grown for 5 days and then induced to differentiate into GC for 20 days. Prior to the assay, cells were washed in DPBS twice and then incubated with DMEM with 2% fatty-acid free BSA (Santa Crus, sc-500949) and Triascin C (5 μM). Cells were treated with forskolin (20 μM) for indicated time periods and supernatant samples were collected. Lipolysis assay was performed with free glycerol reagent (Sigma, F6428-40ML), according to manufacturer instructions.

Thermogenic Dye Assay

ADSCs were induced to differentiate in a 384-well plate as described above. ERThermAC dye (125 nM) was added to the cells in B-8 medium and incubated for 30 minutes at 37° C. in 5% CO2. Cells were washed twice in PBS and fresh B-8 medium was added in the presence or absence of forskolin (10 μM). Cells were imaged immediately and then 3 hours later using an ImageXpress high-content analysis imaging system (Molecular Devices). The average cell intensity was calculated using the ImageXpress acquisition software at the 0 hr and 3 hr timepoints. The percent dye reduction was calculated for each condition (n=24) based on ((I₀ₕ−I₃ₕᵣ)/I₀ₕ)×100, where I is the average intensity.

Results

After confirmation that the GC exhibit a thermogenic adipocyte RNA-seq signature, it was important to establish their functional properties by evaluating the capacity for uncoupled respiration. This was addressed using the Mito Stress Test with a Seahorse XF Analyzer. By comparing O₂ consumption rates (OCR) and extracellular acidification rates (ECAR) of GC and WAs, it is clear that the GC have significantly higher levels of basal and maximal mitochondrial respiration as well as significantly higher levels of glycolysis. (FIGS. 5A and 5B). This difference was increased by addition of forskolin (FSK) (i.e., the mitochondrial respiration and glycolytic responsiveness of beige adipocytes to FSK was significantly greater than that for WA) (FIGS. 5A and 5B). Both basal and maximal respiration were significantly elevated in GC compared to white adipocytes (FIGS. 5C and 5D). Perhaps most importantly, the basal level of uncoupled respiration (proton leak) was considerably (5-fold) higher in GC compared to the WA, and elevated (64.7%) upon FSK treatment relative to basal conditions (FIG. 5E). These data demonstrate that GC are metabolically distinct from WA in vitro and have a metabolic capacity for uncoupled respiration similar to thermogenic adipocytes in vivo (Shinoda, et al., Nat Med 21, 389-394 (2015)).

Thermogenic adipocytes, upon stimulation with forskolin, are known to induce lipolysis and activate their thermogenic programs (Bronnikov et al., J Biol Chem 267, 2006-2013 (1992); Schimmel et al., Am J Physiol 248, E224-229 (1985)). GC were also functionally activated in this manner by forskolin treatment. To establish if increased adenyl cyclase activity activates lipolysis in ADSC-derived beige adipocytes a glycerol release assay was performed. After 4 hours and 8 hours of FSK addition, a significant increase (˜2 and 5-fold, respectively) in the amount of free glycerol in the media was observed, indicating that the GC had undergone lipolysis (FIG. 5F). This confirms that lipolytic activation mechanisms acting through cAMP are intact in ADSC-derived beige cells (FIG. 5F).

Previous studies have demonstrated that FSK induces cAMP and PKA activity to promote lipolysis (Holm, et al., Biochem Soc Trans 31, 1120-1124 (2003)) through a mechanism requiring the phosphorylation of cAMP-response element binding protein (CREB) and P38MAPK activation (Cao, et al., Mol Cell Biol 24, 3057-3067 (2004), Cao, et al., J Biol Chem 276, 27077-27082 (2001)). Increased cAMP levels and PKA activation also activate hormone-sensitive lipase (HSL) by a phosphorylation-dependent mechanism (Ahmadian, et al., Future Lipidol 2, 229-237 (2007)). Activation of HSL is an important determinant of lipolysis in thermogenic adipocytes. To confirm that beige adipocyte activation by FSK occurs by a canonical thermogenic mechanism, HSL, CREB and P38MAPK were investigated by western blot and shown to be phosphorylated within 1 hour of FSK-treatment. This confirms that well-established pathways required for beige adipocyte activation are functional in ADSC-derived beige adipocytes. Overall, these data indicate that ADSC-derived beige adipocytes are activated by the cAMP20 adenyl cyclase signaling axis, resulting in increased uncoupled respiration, glucose utilization and lipolysis.

To assay for thermogenesis, a thermogenic dye (ERthermAC) that undergoes a loss of signal intensity upon thermogenic adipocyte activation (Kriszt et al., Rep 7, 1383 (2017)) was utilized. Following 3 hours of FSK treatment, a consistent reduction in dye signal was observed, indicating that the cells had undergone thermogenesis (FIG. 5G).

Overall these data indicate that the GC are capable of functional activation by FSK stimulation that results in the induction of lipolysis and subsequent thermogenesis.

### Example 3: GC Modulate Metabolic Activity In Vivo

Materials and Methods

Animals

NOD/SCID mice (NOD/ShiLtSz genetic background, Jackson Laboratory, stock no. 001303) were purchased and used to establish a breeding colony. Mice were housed in Tecniplast GM500 individually ventilated cages in a temperature controlled room with 12 hour light/12 hour dark cycles. Only male mice were used for STZ animal studies and females were used in the indirect calorimetry assay, ages dependent on the assay described below.

Animal experiments were performed following IACUC guidelines at the University of Georgia accredited through AAALAC international, and in compliance with Public Health Service policy through NIH Office of Laboratory Animal Welfare and USDA Animal Welfare Act and Regulations.

Transplantations and Thermal Imaging

ADSCs or fully-differentiated ADSC-derived beige adipocytes were transplanted into hindlimb muscles (at 10 locations on both sides) of SCID mice. Approximately 2 millioncells in total were transplanted into each recipient. As an alternative approach, approximately 2 million beige adipocytes were subcutaneously transplanted into the intrascapular region. Thermal imaging was performed using a FLIR A300 infrared camera with ResearchIR software to detect the mean temperature recording.

Indirect Calorimetry

ADSCs or ADSC differentiated to GC for 21 days, were transplanted into hindlimb muscles (at 10 locations on both sides) of 12-week-old SCID female mice with 8 mice per cell type. Approximately 2 million cells in total were transplanted for each host.

Host mice (12 week, female) were housed individually in metabolic cages for 5 days before the transplantation and 7 days after the transplantation with access to food and water ad lithium, 25° C. environment and 12:12-hrs light:dark cycles. Seven control mice and eight experiment mice were used for the analysis. No statistical differences in energy expenditure, oxygen consumption and respiratory exchange ratio were observed prior to transplantations. Metabolic parameters (VO2, VCO2, activity) were measured continuously by an open-circuit indirect calorimetry system (OxyMax, Columbus Instruments) equipped with infrared beam counters. Energy expenditure (EE) were calculated based on the equation: E.E.=(3.815+1.232×RER)×VO2. For histograms, VO2, VCO2, RER, and activity were calculated by averaging values for dark and light cycles separately. Core body temperature and body weight were also recorded weekly.

Results

The results presented demonstrate that GC can be generated at high efficiency from ADSCs and that they are phenotypically, genotypically and metabolically similar to thermogenic adipocytes derived from BAT (Kajimura, et al., Cell Metab 22, 546-559 (2015), Inagaki and Kajimura, Nat Rev Mol Cell Biol 17, 480-495 (2016)). Furthermore, GC can be functionally activated in vitro by FSK.

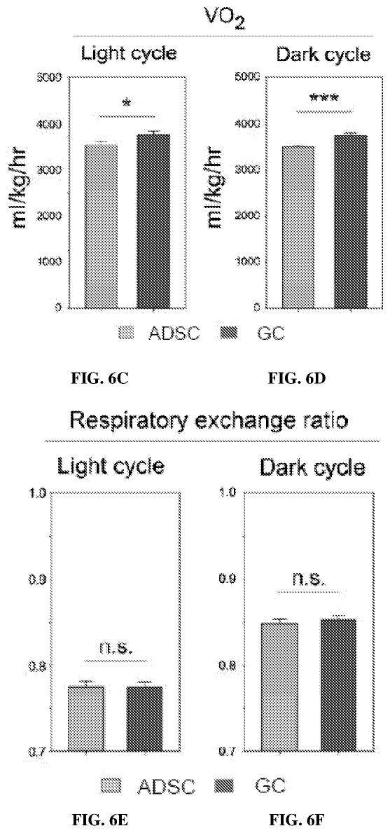

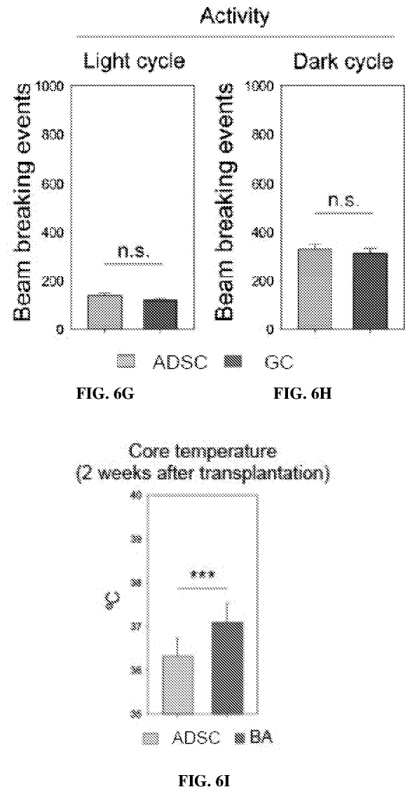

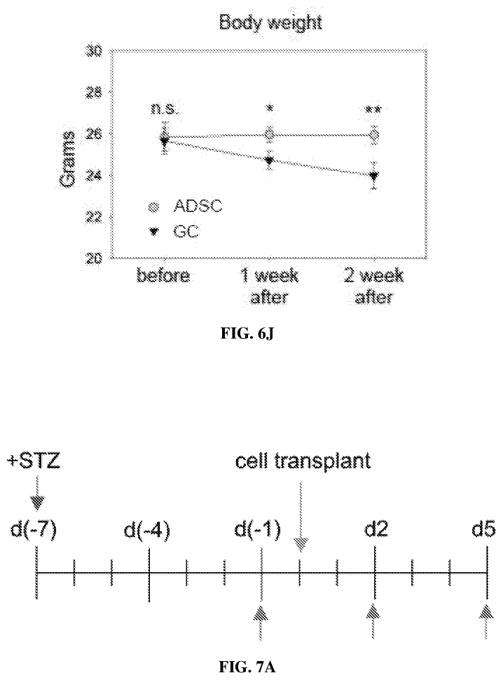

To further investigate the therapeutic potential of GC, murine models were utilized. Following transplantation of GC and ADSCs into the hindlimb muscle of NOD/SCID mice by direct injection, indirect calorimetry was performed to evaluate whole-body energy expenditure. According to the model supported by FIGS. 6A-6J, following transplantation, authentic thermogenic adipocytes would increase total energy expenditure as a consequence of their elevated rates of glucose consumption, fatty acid oxidation and uncoupled respiration. Using metabolic cage assays, this was investigated by measurement of energy expenditure and oxygen consumption. Both parameters were significantly elevated in GC recipients compared to ADSC recipients after 7 days (FIGS. 6A-6B, 6C-6D). Importantly, there were no changes in the respiratory exchange ratio or locomotor activity (beam breaking) levels of the mice, indicating that differences in energy expenditure were not due to increased exercise (FIGS. 6E-6F, 6G-6H).

As a direct calorimetric readout, core body temperatures were measured (FIG. 6I). Mice receiving GC had a ˜2% increase (p<0.005) in core body temperature relative to ADSC recipients, consistent with energy expenditure in GC-transplanted mice (FIG. 6I). Consistent with this, surface body temperatures of mice receiving beige adipocytes were significantly increased over those receiving ADSCs, as determined by thermal imaging. A significant reduction in body weight was also observed at 1 and 2 weeks post-transplantation (FIG. 6J). Water consumption and hydration, food intake and muscle mass was similar in each group of mice, indicating that weight was due to increased caloric consumption. These data provide evidence that GC transplantations result in increased energy expenditure resulting in elevated heat generation and general metabolic activity, as well as a reduction in body weight, and together demonstrate that GC are functional in vivo following transplantation.

### Example 4: GC Modulate Glucose Homeostasis In Vivo

Materials and Methods

STZ-Induced Hyperglycemia

Male NOD/SCID 6-week old mice were intraperitoneally injected with 150 mg/kg streptozotocin (STZ) in an ABSL2 biosafety hood. One week after STZ administration, non-fasting glucose levels in STZ-treated mice were measured by a OneTouch Ultra® 2 Glucose meter. Mice that have non-fasting blood glucose levels exceeding 250 mg/dL were included in the study and semi-randomly separated into control and experimental groups. The grouping ensures that the non-fasting blood glucose levels and body weight have no significant difference before the transplantation.

ADSCs or ADSC differentiated to GC for 21 days, were transplanted into hindlimb muscles on both sides with 8 mice per cell type. Approximately 2 million cells in total were transplanted for each host. Blood glucose levels were measured daily for 7 days and mean values were graphed as box plots to indicate total changes.

MicroPET

3.7 MBq of 18F-FDG was injected via the tail vein and an acquisition scan was performed 1 hour post injection. Isolflurane anesthesia were used on mice throughout the injection and imaging processes. A region of interest (ROI) were drawn over the injection sites and the concentration of average radioactivity was determined based on the mean pixel values within the ROI volume and converted to counts per ml per minute (counts/ml/min). Assuming a tissue density of 1 g/ml, the counts/ml/min was converted to counts/gram/min and divided by the injected dose (ID) to obtain an imaging ROI-derived % ID/g. Four mice were used for each experimental group.

Results

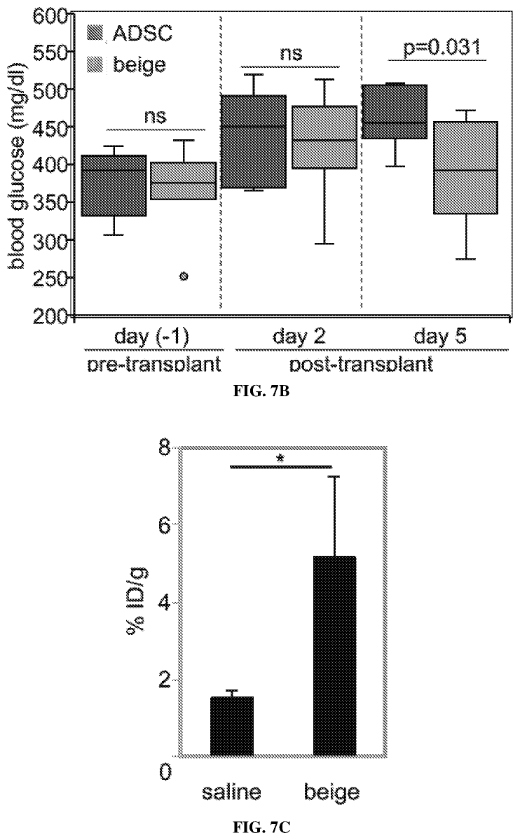

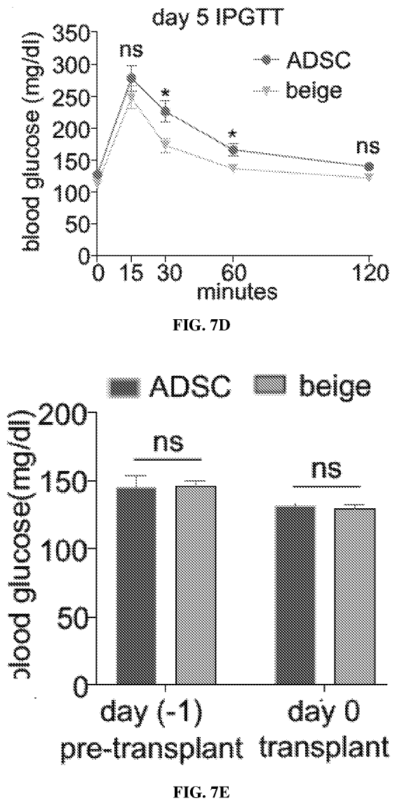

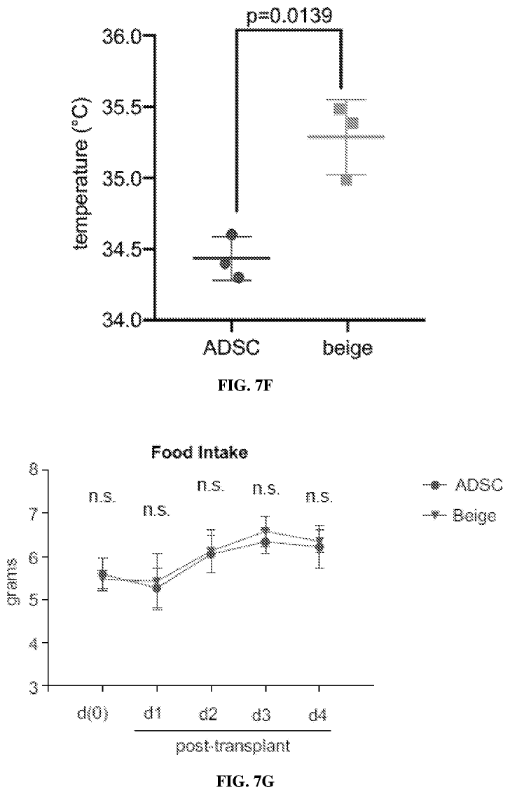

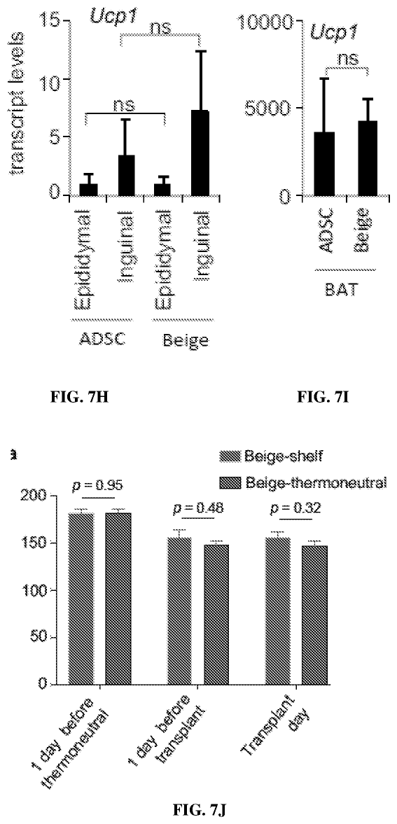

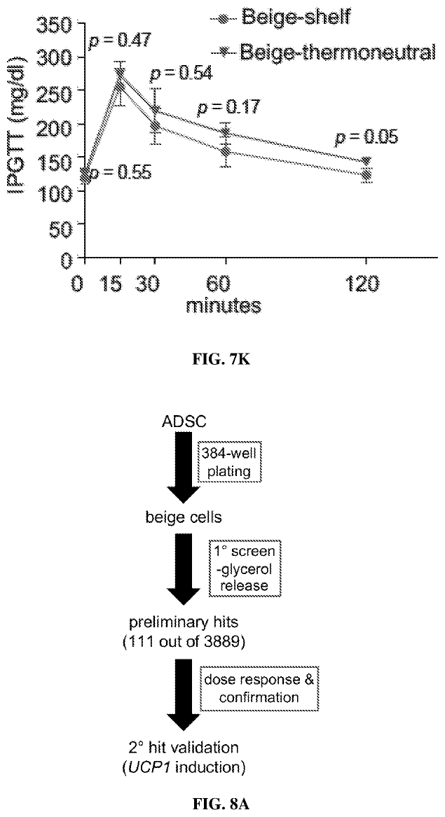

Thermogenic adipocyte depots play important roles in metabolic homeostasis by improving the clearance of circulating glucose and triglycerides (Harms, et al., Nat Med, 19, 1252-1263 (2013), Lidell, et al., J Intern Med 276, 364-377 (2014), Singh and Dalton, Trends Endocrinol Metab, 29, 349-359 (2018). To determine if transplanted GC are capable of partially clearing circulating glucose, they were transplanted into a chemically-induced (streptozotocin, STZ) hyperglycemic mouse model. Male NOD/SCID mice were treated with STZ and then transplanted with either ADSCs or GC into the hindlimb muscle. Non-fasting blood glucose levels were then monitored daily for one week (FIG. 7A, 7B). Over the time-course of this experiment, beige cells significantly reduced the levels of circulating glucose in hyperglycemic mice compared to ADSC controls (p<0.05) (FIG. 7B)

To further examine the in vivo functionality of the transplanted GC, infrared thermal imaging was performed. Compared to ADSC-control transplants, GC exhibited increased thermogenesis following transplantation, which further validated the function of the cells in vivo.

To confirm that transplanted GC clear circulating glucose, microPET imaging of mice transplanted metabolically labelled by systemic infusion of ¹⁸F-FDG was performed. GC-transplanted mice had a consistent increase in the amount of radiolabeled glucose taken up by the cells. See, e.g., FIG. 7C, which shows a ˜3.5-fold increase in 18F-FDG uptake was observed in beige cells compared to controls (p<0.05). This was supported by intraperitoneal glucose tolerance tests (IPGTT) where mice transplanted with beige adipocytes cleared circulating glucose significantly more quickly (p<0.05) than ADSC controls (FIG. 7D, 7E).

Differences in food intake (FIG. 7G) or potential ‘browning’ of white adipose depots (FIG. 7H, 7I) do not account for changes in metabolic activity observed between beige and ADSC recipient mice. Under thermoneutral conditions, glucose clearance in beige cell recipients was not statistically different to ADSC-controls, although a positive trend was noted (FIG. 7J, 7K). This indicates that transplanted beige cells are likely to be under similar physiological regulatory cues that regulate thermogenic adipocytes in vivo (Clayton, et al., Am J Physiol Regul Integr Comp Physiol 315, R627-R637 (2018)).

Increased glucose clearance in beige adipocyte recipients was accompanied by increased thermogenic activity (FIG. 7F). Histological analysis of tissue isolated from these mice confirmed the presence of adipocytes with multilocular lipid droplets, consistent with the presence of beige adipocytes. This morphology is clearly different to subcutaneous white adipocyte depots from the same mouse Immunohistochemistry analysis of the transplanted (DiI+) tissue confirmed the engraftment of human UCP1+ cells with multilocular lipid droplets. Human UCP1+ cells were absent in sub-cutaneous white adipose depots.

These data are indicative of the integration of metabolically-active GC into host muscle. These data show that GC transplantations have the ability to uptake glucose and modulate glucose levels in a chemically-induced diabetic mouse model. Thus, these data demonstrate that human ADSC-derived beige cells engraft into recipient mice and exhibit metabolic activity consistent with them being functional beige adipocytes.

### Example 5: A High-Throughput Drug Screen Identifies Regulators of Beige Adipocyte Activity

Materials and Methods

High-Throughput Drug Screening

ADSCs were plated in 384-well plates at a density of 5000 cell/cm2 and grown to confluency over ˜4 days. Cells were then differentiated to beige adipocytes using B-8 medium for 21 days. All cell culture and incubations in the 384-well format were done at 37° C. and 5% CO₂. Compounds from the library of pharmacologically active compounds (LOPAC, Sigma) and the Emory Enriched Bioactive Library (Mo, et al., Cell Chem Biol. 26(3):331-339.e3 (2019). doi: 10.1016/j.chembiol.2018.11.011) (3889 in total) were added at a final concentration of 20 μM. The LOPAC library are composed of 1280 biologically active compounds and impacts most signaling pathways and drug classes, including cell signaling, phosphorylation, cell stress, ion channels, G-Protein Coupled Receptors, hormone and cell cycle regulators. The Emory Enriched Bioactive Library consisted of 2609 compounds including 1018 FDA-approved compounds (SelleckChem) that affect over 20 signaling pathways. The use of FDA-approved drugs may allow for re-purposing for therapeutic use.

Lipolysis assays were performed after 24 hours of drug treatment by measuring free glycerol released in the 384-well format, using the procedure described above. Absorbance for each compound was normalized to untreated control samples for each plate and calculated as a percent of the control. Cut-offs for “activating” and “inhibiting” compounds were set at 2-fold. Validation assays were performed for select compounds at a range of concentration doses.

Results

The identification of new drugs and regulatory pathways that promote the activation of endogenous thermogenic adipocytes may result in the development of new therapeutics for the treatment of metabolic diseases, such as diabetes and/or obesity. To test the utility of beige adipocytes for drug discovery, the LOPAC and Emory Enriched Bioactive Library (Mo, et al., Cell Chem Biol. 26(3):331-339.e3 (2019). doi: 10.1016/j.chembio1.2018.11.011) (FIG. 8A) were screened using a lipolysis assay (glycerol release) in a 384 well plate format. Out of 3889 compounds evaluated in the primary screen, 111 compounds significantly stimulated glycerol release and were subject to further evaluation (FIG. 8B).

Importantly, well-known activators of thermogenic adipocytes including FSK and β-adrenergic agonists such as isoproterenol were identified as primary hits in this screen (FIG. 8B, Table 8), confirming the utility of the assay. Inhibitors of p38 MAPK and adrenoreceptors that suppress lipolysis were also identified (Singh, et al., Cell Stem Cell 10, 312-326 (2012), Wang, et al., Blood 110, 4111-4119 19 (2007), Cliff, et al., Cell Stem Cell 21, 502-516 e509 (2017).) (FIG. 8B, Table 8).

TABLE 8
Representative compounds are shown with relative levels of glycerol
release (% control) and reported targets for these compounds.

| Compound | % Control | Target |
|---|---|---|
| (±)-Isoproterenol | 389.02 | Beta Adrenoreceptor |
| hydrochloride | | |
| S(−)-Isoproterenol (+)- | 367.84 | Beta Adrenoreceptor |
| bitartrate | | |
| R(+)-Isoproterenol (+)- | 363.04 | Beta Adrenoreceptor |
| bitartrate | | |
| Forskolin | 262.31 | Adenylate Cyclase |
| Mirabegron | 211.65 | Adrenoreceptor Agonist |
| PD 169316 | 39.64 | P38 MAPK inhibitor |
| PH-797804 | 38.23 | P38 MAPK inhibitor |
| SB202190 (FHPI) | 34.88 | P38 MAPK inhibitor |
| Carteolol HLC | 32.31 | Adrenoreceptor Antagonist |
| RX 821002 hydrochloride | 30.77 | Adrenoreceptor Antagonist |

The chemical enrichment analysis tool, BiNChE (Moreno, et al., BMC Bioinformatics 16, 56 (2015)) was then used to identify mechanistic links between the 111 preliminary hits identified in this screen. Adrenergic signaling was identified as a key regulatory pathway for beige adipocyte activation in the screen (FIG. 8C). Thirty-six of the primary screen hits are known adrenergic agonists, indicating that the screening platform is reliably detecting authentic activators of beige adipocytes.

Next, lipolysis assays were performed for all 111 compounds identified in the primary screen at three different concentrations, of which twenty-four were selected for further analysis based on their newness and/or reproducibility. All twenty-four compounds significantly increased lipolysis in the three replicates, confirming the activity of these compounds in the primary screen (Table 9).

TABLE 9
Results of Compounds in Lipolysis Assays

| | Conc. | % of | % of | % of | % of | | |
|---|---|---|---|---|---|---|---|
| Sample | (mM) | Control (1) | Control (2) | Control (3) | Control (4) | Target | Category |
| Negative | 0 | 78.21 | 82 | 62.42 | 68.39 | | |
| | 0 | 76.9 | 70.59 | 85.93 | 110.23 | | |
| | 0 | 62.98 | 79.65 | 89.23 | 49.2 | | |
| Forskolin | 2.2 | 416.67 | 393.76 | 251.65 | 247.36 | cAMP | Epigenetics |
| | 6.7 | 311.19 | 362.24 | 241.1 | 241.95 | | |
| | 20 | 416.67 | 330.12 | 252.97 | 267.24 | | |
| Isoproterenol | 2.2 | 282.38 | 272 | 236.26 | 251.38 | beta | Sympathomimetic |
| HCl | | | | | | | amine acting |
| | 6.7 | 283.57 | 294.24 | 240.99 | 235.75 | | almost exclusively |
| | 20 | 288.57 | 268 | 251.87 | 237.01 | | on beta |
| | | | | | | | adrenoceptors; |
| | | | | | | | bronchodilator; |
| | | | | | | | active enantiomer |
| | | | | | | | of Isoproterenol |
| Mirabegron | 2.2 | 142.5 | 197.53 | 172.2 | 131.15 | Adrenergic | Others |
| | | | | | | Receptor | |
| | 6.7 | 202.62 | 161.88 | 168.46 | 192.3 | | |
| | 20 | 200 | 211.41 | 65.05 | 200.8 | | |
| Isoetharine | 2.2 | 293.69 | 307.41 | 258.13 | 106.21 | Adrenergic | Others |
| Mesylate | | | | | | Receptor | |
| | 6.7 | 372.74 | 300.59 | 338.79 | 247.47 | | |
| | 20 | 416.67 | 304.12 | 204.18 | 260.34 | | |
| Metaproterenol | 2.2 | 327.14 | 297.53 | 291.21 | 304.83 | Adrenergic | Others |
| Sulfate | | | | | | Receptor | |
| | 6.7 | 310.83 | 411.76 | 292.53 | 283.1 | | |
| | 20 | 305.36 | 292.71 | 308.35 | 274.25 | | |
| Phenylephrine | 2.2 | 338.33 | 252.94 | 220.77 | 239.89 | Adrenergic | Others |
| HCl | | | | | | Receptor | |
| | 6.7 | 271.43 | 266.24 | 240.44 | 231.72 | | |
| | 20 | 353.33 | 372.82 | 248.02 | 231.84 | | |
| Lofexidine | 2.2 | 104.17 | 115.29 | 118.79 | 91.38 | Adrenergic | Others |
| HCl | | | | | | Receptor | |
| | 6.7 | 121.9 | 176.47 | 180 | 148.85 | | |
| | 20 | 204.52 | 199.65 | 147.8 | 191.49 | | |
| Nanchangmycin | 2.2 | 259.29 | 209.65 | 192.42 | 124.48 | Others | Metabolism |
| | | | | | | (antibiotic) | |
| | 6.7 | 196.07 | 104.94 | 147.69 | 106.09 | | |
| | 20 | 147.14 | 168.94 | 98.9 | 168.85 | | |
| Acetanilide | 2.2 | 316.67 | 377.41 | 251.1 | 119.31 | Others | Neuronal |
| | | | | | | | Signaling |
| | 6.7 | 252.38 | 332.24 | 246.15 | 249.31 | | |
| | 20 | 298.33 | 295.18 | 252.86 | 257.59 | | |
| SU 5416 | 2.2 | 280.6 | 378.59 | 214.4 | 231.61 | VEGFR | Potent and |
| | | | | | | PTK | selective VEGFR |
| | 6.7 | 349.29 | 366.35 | 251.1 | 238.39 | | PTK inhibitor; |
| | | | | | | | inhibits VEGF- |
| | | | | | | | induced |
| | | | | | | | angiogenesis |
| | 20 | 282.86 | 266.94 | 232.53 | 246.32 | | |
| Levosimendan | 2.2 | 214.52 | 163.41 | 189.23 | 235.63 | Calcium | Others |
| | | | | | | sensitizer | |
| | 6.7 | 197.14 | 189.41 | 210.77 | 245.52 | | |
| | 20 | 260.71 | 279.65 | 246.81 | 339.89 | | |
| PIK-75 | 2.2 | 197.74 | 134.71 | 183.08 | 136.9 | DNA- | DNA Damage |
| | | | | | | PK, PI3K | |
| | 6.7 | 280.95 | 197.53 | 165.05 | 207.93 | | |
| | 20 | 158.45 | 244.82 | 198.79 | 233.79 | | |
| MLN9708 | 2.2 | 251.07 | 138.59 | 204.51 | 114.25 | Proteasome | JAK/STAT |
| | 6.7 | 197.74 | 228.35 | 175.93 | 115.4 | | |
| | 20 | 137.5 | 173.18 | 117.25 | 90 | | |
| Anagrelide | 2.2 | 125.12 | 146.24 | 124.18 | 144.14 | PDE III | Phosphodiesterase |
| HCl | | | | | | | III inhibitor |
| | 6.7 | 194.52 | 267.76 | 191.32 | 156.21 | | |
| | 20 | 196.19 | 182.94 | 211.87 | 160.92 | | |
| PF-04691502 | 2.2 | 171.55 | 138.35 | 232.86 | 105.98 | Akt, mTOR, | Angiogenesis |
| | | | | | | PI3K | |
| | 6.7 | 257.98 | 216.59 | 225.05 | 155.98 | | |
| | 20 | 304.52 | 224.71 | 171.76 | 302.07 | | |
| Felodipine | 2.2 | 242.74 | 219.65 | 202.75 | 206.21 | L-type | L-type calcium |
| | | | | | | | channel blocker |
| | 6.7 | 283.69 | 241.41 | 227.91 | 255.75 | | |
| | 20 | 291.07 | 274.35 | 244.07 | 244.94 | | |
| LY-310, 762 | 2.2 | 179.4 | 180.24 | 180.44 | 143.79 | 5-HT1D | Potent, selective |
| | | | | | | | 5-HT 1D |
| | | | | | | | serotonin receptor |
| | | | | | | | antagonist. |
| | 6.7 | 179.76 | 199.88 | 222.09 | 289.77 | | |
| | 20 | 264.29 | 223.06 | 225.27 | 201.38 | | |
| Pilocarpine | 2.2 | 108.93 | 134.94 | 176.37 | 89.77 | Muscarinic | Nonselective |
| nitrate | | | | | | | muscarinic |
| | | | | | | | acetylcholine |
| | | | | | | | receptor agonist |
| | 6.7 | 219.64 | 259.53 | 167.36 | 84.25 | | |
| | 20 | 199.88 | 289.53 | 229.67 | 180.92 | | |
| Trequinsin | 2.2 | 239.88 | 250.59 | 188.35 | 244.25 | PDE III | Phosphodiesterase |
| HCl | | | | | | | III (PDE III) |
| | | | | | | | inhibitor |
| | 6.7 | 271.19 | 263.53 | 231.98 | 214.94 | | |
| | 20 | 284.29 | 291.65 | 256.26 | 247.59 | | |
| Sanguinarine | 2.2 | 179.4 | 142.35 | 71.32 | 121.03 | Na+/K+ | Inhibitor of Mg2+ |
| | | | | | | ATPase | and Na+/K+- |
| | 6.7 | 218.33 | 198 | 128.13 | 101.15 | | ATPase; isolated |
| | | | | | | | from the leaves |
| | | | | | | | and stems of |
| | | | | | | | Macleaya cordata |
| | | | | | | | and microcarpa |
| | 20 | 184.88 | 155.29 | 132.64 | 160.34 | | |
| AT9283 | 2.2 | 162.86 | 114.71 | 142.2 | 132.53 | JAK, Bcr- | DNA Damage |
| | | | | | | Abl, Aurora | |
| | | | | | | Kinase | |
| | 6.7 | 150.71 | 152.47 | 160.88 | 177.24 | | |
| | 20 | 131.9 | 144.12 | 201.87 | 158.74 | | |
| Milrinone | 2.2 | 99.05 | 116.71 | 126.04 | 169.89 | PDE III | Phosphodiesterase |
| | | | | | | | III inhibitor |
| | 6.7 | 235.95 | 153.88 | 116.15 | 142.41 | | |
| | 20 | 167.98 | 204.94 | 170.66 | 184.94 | | |
| Dinaciclib | 2.2 | 174.88 | 231.53 | 193.19 | 178.62 | CDK | MAPK |
| | 6.7 | 180.36 | 203.76 | 149.12 | 128.85 | | |
| | 20 | 291.19 | 246.35 | 60.66 | 261.95 | | |
| L-368, 899 | 2.2 | 196.9 | 128 | 118.57 | 91.72 | Oxytocin | Non-peptide |
| | | | | | | receptor | oxytocin receptor |
| | | | | | | | antagonist. |
| | 6.7 | 185.48 | 151.88 | 130.11 | 148.51 | | |
| | 20 | 143.81 | 179.29 | 192.97 | 166.21 | | |

To determine if hits identified in the first-round screen activated the thermogenic program, UCP1 transcript levels were used as a readout. This secondary screen incorporated adrenergic agonists, such as Mirabegron, isoproterenol and phenylephrine, and eighteen additional compounds with no reported function in thermogenic regulation (FIG. 8D, 8E, 8F). 18/25 (72%) of the lipolysis-activating compounds identified in the primary screen also activate the thermogenic program through UCP1. Twelve of these compounds have no reported adrenergic agonist activity and could potentially operate through different signaling mechanisms (FIG. 8D and Table 9).

Of note, two phosphodiesterase III (PDE III) inhibitors were identified as activators in both rounds of screening. Other classes of compounds include receptor/channel antagonists (L-368,899, pilocarpine nitrate, LY310-762, Felodipine) and protein kinase inhibitors (Dinaciclib, AT9283, PF-04691502, PIK-75, SU 5416). These data also demonstrate the broad usefulness of beige adipocytes as a platform for drug discovery and hit/target validation studies.

Current technologies for generating thermogenic adipocytes are limited by their reliance on genetic modifications (Liu, et al., Cell Res 23, 851-854 (2013)), low efficiency due to the use of serum and embryoid bodies (Hafner, et al., Sci Rep 6, 32490 (2016), Mohsen-Kanson, et al., Stem Cells 32, 1459-1467 (2014)). Moreover, there is an absence of data regarding the functional validation of these cells in animal models (Su, et al., Cell Rep 25, 3215-3228 e3219 (2018)). The use of thermogenic adipocytes derived from induced pluripotent stem cells for autologous transplantations is also hindered due to low purity and lengthy reprogramming and/or differentiation protocols (Koksharova, et al., Diabetes Technol Ther 19, 96-102 (2017), Cannon & Nedergaard, Physiol Rev 84, 277-359 (2004), Ahfeldt, et al., Nat Cell Biol 14, 209-219 (2012)). The technology described herein solves many of the existing problems associated with the use of human thermogenic adipocytes for therapeutic development.

By utilizing a fully-defined, serum-free differentiation medium in conjunction with ADSCs, GC can be generated over a 21 day period with purity that exceeds 90%, a time-frame that is compatible with therapeutic development. GC exhibit a molecular profile that is consistent with them being authentic beige adipocytes. Moreover, these cells have metabolic characteristics consistent with them having thermogenic activity, including FSK-responsive uncoupled respiration and lipolysis. Finally, using two independent in vivo models, GC are shown to function in vivo by increasing energy consumption, promote a decline in body mass and improve glucose homeostasis. This is the first time that human ADSC-derived thermogenic adipocytes have been shown to provide function in vivo, which has potential utility in the treatment of metabolic diseases.

Thus, GC can be used as therapeutics to treat metabolic disease or for disease modeling. For example, GC could be used as a cell-based therapy for treating diseases, such as type 2 diabetes. As insulin resistance and obesity are common problems amongst T2D patients, utilizing GC as a cell-based therapeutic to modulate glucose, triglyceride homeostasis and body weight provides a viable strategy to treat this disease.

An additional advantage that ADSC-derived beige cells have over the use of pluripotent cells is that the former can be easily isolated from liposuction aspirates of peripheral white adipose. This can potentially generate a large supply of beige adipocytes for autologous transplantation in a relatively short time-frame, alleviating problems associated with genomic drift during amplification and thereby providing a safe, transplantable cell source. Over 150 clinical trials have described the efficacious and safe use of ADSCs (clinicaltrials website), thereby demonstrating their usefulness in a therapeutic setting. A therapeutic pipeline for ADSC-derived beige cells may only need an additional differentiation procedure prior to transplantation.

As an alternative approach, an “off the shelf” allogeneic beige adipocyte cell therapy could involve immune tolerant scaffolds (Headen, et al., Nat Mater 17, 732-739 (2018), Skoumal, et al., Biomaterials 192, 271-281 (2019)).

Use of beige adipocyte in a transplant setting could potentially follow a similar approach used for β-cell and pancreatic progenitor cell transplants into T1D patients, where encapsulation in a protective device is used (Agulnick, et al., Stem Cells Transl Med 4, 1214-1222 (2015), Vegas, et al., Nat Med 22, 306-311 (2016)). The omental pouch (Rjab, et al., Curr Diab Rep 10, 332-337 (2010)) and subcutaneous sites (Agulnick, et al., Stem Cells Transl Med 4, 1214-1222 (2015)) have been proposed as suitable transplantation sites due to their potential for neo-vascularization around grafts. High-efficacy has been reported for islet transplants into the omental pouch in diabetic rodent models, canine models and non-human primate models (al-Abdullah, et al., Cell Transplant 4, 297-305 (1995), Ao, et al., Transplantation 56, 524-529 (1993), Berman, et al., Am J Transplant 9, 91-104 (2009), Gustayson, et al., Am J Transplant 5, 2368-2377 (2005), Kin, et al., Am J Transplant 3, 281-285 (2003)) and subcutaneous transplantation of devices into patients is in clinical trials (Agulnick, et al., Stem Cells Transl Med 4, 1214-1222 (2015), Vegas, et al., Nat Med 22, 306-311 (2016)). Both represent potential sites for beige cell transplantations.

Second, GC could serve as a source of secreted factors (adipokines) or extracellular vesicles, such as exosomes, that may have therapeutic usefulness (Chen et al., Nat Commun 7, 11420 (2016); Thomou et al., Nature 542, 450-455 (2017)).

Third, GC are a useful cell source for performing drug screening applications. Since this is a fully-defined and serum-free system, factors/inhibitors may be removed or added to modulate the differentiation process in a high-throughput screening approach to look for compounds that promote or activate beiging and thermogenesis.

A wide-range of drugs are currently used for the treatment of T2D but none specifically target thermogenic adipocytes as a means of controlling energy expenditure and circulating levels of glucose and triglycerides. A desired effect for T2D drugs is to normalize levels of circulating blood-glucose (A1C <7.0). This can be achieved by targeting different cell types and regulatory mechanisms and where treatment is dependent on drug efficacy and contraindications (American Diabetes, A. Standards of Medical Care in Diabetes-2019 Abridged for Primary Care Providers. Clin Diabetes 37, 11-34 (2019)). Most T2D drugs however, have restricted target activity and efficacy thus highlighting the need to develop additional therapeutic approaches.

Metformin, the first-line orally administered therapeutic for T2D, primarily functions by inhibiting gluconeogenesis in the liver (American Diabetes, A. 9. Pharmacologic Approaches to Glycemic Treatment: Standards of Medical Care in Diabetes-2019. Diabetes Care 42, S90-S102 (2019)) but does not specifically target thermogenic adipocytes, insulin secretion or carbohydrate absorption. In contrast, sulfonureas, dipeptidyl-peptidase inhibitors and meglitinides modulate glucose levels by promoting insulin secretion. Another approach used clinically is to administer inhibitors of α-glucosidases which decrease intestinal carbohydrate digestion and absorption rates. Nearly all of these drugs have potential side-effects including weight gain, gastrointestinal effects and hypoglycemia and importantly, none of them target thermogenic adipocytes. Development of drugs targeting thermogenic adipocytes could have widespread application among T2D patients and for the prevention of disease progression in pre-diabetics (A1C: 5.7-6.4), where there are no FDA-approved medications as a sole-treatment option (American Diabetes, A. Standards of Medical Care in Diabetes-2019 Abridged for Primary Care Providers. Clin Diabetes 37, 11-34 (2019)).

The proof-of-concept drug screening described herein makes drug development in this area feasible. Identification of multiple β-adrenergic agonists in the HTS validated the cell platform being used for further library screening and therapeutic development. Although β3-adrenergic receptor ((33-AR) agonists can potentially target human thermogenic adipocytes, this idea is primarily based on murine studies (Cannon, et al., Physiol Rev 84, 277-359 (2004) (Jimenez, et al., Eur J Biochem 270, 699-705 (2003), Nahmias, et al., EMBO J 10, 3721-3727 (1991)). The role of β-adrenergic signaling is less clear in humans (Mund, et al., Cardiol Rev 21, 265-269 (2013)), partly because β3-AR agonists show no significant impact on energy expenditure in clinical trials (Larsen, et al., Am J Clin Nutr 76, 780-788 (2002), Redman, et al., J Clin Endocrinol Metab 92, 527-531 (2007), Weyer, et al., Diabetes 47, 1555-1561 (1998)). Only one agonist linked to β3-AR signaling (Mirabegron) was shown to have efficacy in the HTS while β1-AR, β2-AR or β-AR agonists were identified. Although β1-AR and β2-AR agonists show efficacy, they also have known roles in regulation of cardiovascular function (al-Abdullah, et al., Cell Transplant 4, 297-305 (1995)). The same may also be true for β3-AR agonists because the β3-AR is expressed in a broader range of tissues than initially thought (Alexandre, et al., Br J Pharmacol 173, 415-428 (2016)).

The identification of non-β-adrenergic mechanisms that activate thermogenic adipocytes highlights the desire to identify new targets and develop alternative strategies for development of therapeutics that target brown and beige cells (Chen, et al., Nature 565, 180-185 (2019), Ye, et al., Proc Natl Acad Sci USA 110, 12480-12485 (2013)). HTS using beige cells is likely to be a viable approach to identify drugs that can address some of the therapeutic problems associated with treatment of T2D. In total, twelve new compounds were identified that activated the thermogenic program, as determined by UCP1 induction and lipolytic activity. There is no evidence however, that links these compounds to β-adrenergic signaling so they potentially impact thermogenic adipocytes through non-β-adrenergic mechanisms. PDE III inhibitors (Trequinsin and Anagrelide) were identified as having efficacy in beige cell activation. Links between PDE III and the activity of thermogenic adipocytes have not been reported, although inhibition of PDE IV in mouse primary brown adipocytes activates thermogenesis through a PKA-dependent mechanism (Braun, et al., J Exp Biol 221 (2018), Larsson, et al., Cell Signal 28, 204-213 (2016)). No links between these compounds and metabolic disease have been established, indicating that use of ADSC-derived beige adipocytes has usefulness as a platform for drug discovery in this area. Further testing is now required to establish the efficacy of compounds identified here in the modulation of metabolic activity in vivo.

Fourth, a GC differentiation system can be used with patient samples for autologous transplantation or to model diseases. For example, the presence of thermogenic adipocytes are known to be considerably reduced in obese individuals (Cypess et al., N Engl J Med 360, 1509-1517 (2009); Saito et al., Diabetes 58, 1526-1531 (2009); van Marken Lichtenbelt et al., N Engl J Med 360, 1500-1508 (2009); Virtanen et al., N Engl J Med 360, 1518-1525 (2009)). A differentiation system could therefore be used to evaluate differences in functional activity between GC established from lean or obese individuals, in order to identify genetic variants and polymorphisms related to obesity or obesity-associated diseases.

In sum, the technology disclosed herein provides a range of opportunities for studying, and intervening in, metabolic diseases.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A thermogenic adipocyte comprising
(i) increased uncoupling protein 1 (UCP1) expression, increased Type II iodothyronine deiodinase (DIO2) expression, or a combination thereof relative to naturally-occurring Adipose-derived stem cells (ADSC), white adipocytes (WAT), or a combination thereof; and
(ii) reduced Intercellular Adhesion Molecule 1 (ICAM1) expression, increased matrix metalloproteinase-3 (MMP3) expression, or a combination thereof relative to naturally-occurring brown adipocytes (BAT), beige cells, or a combination thereof.

2. The thermogenic adipocyte of claim 1 comprising increased UCP1 and DIO2 expression.

3. The thermogenic adipocyte of claim 1 comprising reduced ICAM1 and increased MMP3 expression relative to naturally-occurring brown adipocytes (BAT), beige cells, or a combination thereof.

4. The thermogenic adipocyte of claim 1, wherein the expression comprises mRNA expression, protein expression, or the combination thereof.

5. The thermogenic adipocyte of claim 1, wherein UCP1 expression, DIO2 expression, or the combination thereof is further increased following contacting the cell with forskolin.

6. The thermogenic adipocyte of claim 1, where levels of mitochondrial respiration, glycolysis, or a combination thereof in the cell is increased relative to WAT.

7. The thermogenic adipocyte of claim 6 wherein the respiration is basal respiration, maximal respiration, or a combination thereof.

8. The thermogenic adipocyte of claim 6, wherein the respiration is uncoupled respiration (proton leak).

9. The thermogenic adipocyte of claim 6, wherein the respiration, glycolysis, or combination thereof in the cell is further increased following contacting the cell with forskolin.

10. The thermogenic adipocyte of claim 1, wherein contacting the cell with forskolin induces or increases lipolysis or activates a thermogenic program.

11. The thermogenic adipocyte of claim 10, wherein the lipolysis comprises an increase in extracellular glycerol.

12. The thermogenic adipocyte of claim 10, wherein activation of a thermogenic program corresponds with an increase in the cells ability to reduce signal intensity of a thermogenic dye.

13. The thermogenic adipocyte of claim 1, wherein the cell can elevate energy expenditure and/or oxygen consumption in a subject following transplantation therein, preferably without a change in the respiratory exchange ratio and/or locomotor activity in the subject.

14. A serum-free cell differentiation media comprising one or more growth factors and/or inhibitors selected from
(a) BMP7,
(b) IGF-1,
(c) FGF2,
(d) Y-27632,
(e) Rosiglitazone,
(f) Dexamethasone,
(g) Triiodo-L-thyronine (LT3), and
(h) isobutylmethylxanthine (IBMX);
optionally in combination with one or more of the additives selected from
(i) bovine serum albumin (BSA) (e.g., Probumin®)
(ii) one or more antibiotics and/or antimycotics (e.g., Gibco® Antibiotic-Antimycotic solution which includes penicillin, streptomycin, and Gibco Amphotericin B)
(iii) one or more non-essential amino acids (e.g., L-alanine, L-asparagine, L-aspartic acid, L-glycine, L-serine, L-proline and L-glutamic acid)
(iv) L-glutamine e.g., a stabilized dipeptide form thereof such as Corning® Glutagro™ Supplement
(v) one or more components of Trace Elements A (e.g., cupric sulfate, ferric citrate, sodium selenite, zinc sulfate, and combinations thereof)
(vi) one or more components of Trace Elements B (e.g., ammonium molybdate, ammonium vanadate, manganese sulfate, nickel sulfate, sodium silicate, stannous chloride, hydrochloric acid, and combinations thereof)
(vii) one or more components of Trace Elements C (e.g., aluminum chloride, barium acetate, cadmium chloride, chromic chloride, cobalt dichloride, germanium dioxide, potassium bromide, potassium iodide, rubidium chloride, silver nitrate, sodium fluoride, zirconyl chloride, and combinations thereof)
(viii) L-ascorbic acid, and
(ix) Transferrin;
in a base media comprising a balanced salt solution, amino acids, and vitamins, wherein the media can induce differentiation of ADSC, WAT, or both into thermogenic adipocytes.

15.-19. (canceled)

20. A cell culture media comprising the ingredients according to

| | | Final | |
|---|---|---|---|
| Compound | Diluent | Conc. | |
| Probumin ® | DMEM/F-12 | 2% | |
| Pen/Strep/anti- | — | 1X | |
| mycotic | | | |
| Nonessential amino | — | 1X | |
| acids | | | |
| GlutaGro ™ | — | 1X | |
| Trace Elements A | — | 1X | |
| Trace Elements B | — | 1X | |
| Trace Elements C | — | 1X | |
| L-ascorbic acid | ddH2O | 50 | ug/ml |
| Transferrin | DMEM/F-12 | 10 | ug/ml |
| DMEM/F-12 | — | 1X | |
| BMP7 | 4 mM | 100 | ng/ml |
| | HCL + 0.2% Probumin | | |
| IGF-1 | 4 mM | 200 | ng/ml |
| | HCl + 0.2% BSA | | |
| FGF2 | PBS + 0.2% BSA | 8 | ng/ml |
| Y-27632 | DMSO | 10 | μM |
| Rosiglitazone | DMSO | 2 | μM |
| Dexamethasone | DMSO | 1 | μM |
| Triiodo-L-thyronine | DMSO | 1 | nM |
| (LT3) | | | |
| IBMX | DMSO | 500 | μM |

21. A method of making thermogenic adipocytes comprising incubating ADSC or WAT with media of claim 14 until the cells differentiate into thermogenic adipocytes.

22.-23. (canceled)

24. A cell made according to the method of claim 21.

25. Conditioned media made according a method comprising incubating the cells of claim 1 in a tissue culture media, optionally a serum-free tissue culture media, for one or more days and separating the media from the cells.

26.-33. (canceled)

34. A method of treating a subject in need thereof comprising administering the subject an effective amount of the cells of claim 1.

35.-39. (canceled)

40. A method of treating one or more diseases, disorders, or conditions selected from obesity or excessive weight gain, metabolic disorders, vascular disease, heart disease, atherosclerosis, dyslipidemia, liver steatosis, obesity or excessive weight gain, loss of physical activity, and loss of endurance comprising administering the subject an effective amount of a composition comprising one or more active agents selected from Trequinisn HCL, Anagrelide, Milrinone, L-368,899, pilocarpine nitrate, LY310-762, Felodipine, Dinaciclib, AT9283, PF-04691502, PIK-75, SU 5416, and pharmaceutically acceptable salts thereof.

41.-47. (canceled)

48. A method of screening compounds for an effect on the cells of claim 1 comprising adding one or more compounds to the thermogenic adipocytes in vitro and analyzing the molecular and/or functional effect of the compound on the cells.

49.-54. (canceled)
